SEQUENCE LISTING (2 sequences)

SEQ ID NO: 1 - protein, 56 aa, Artificial Sequence
  a compound for targeting and treating a spinal fusion site
  SITE (29)..(29): Where “X” at position 29 is alpha- aminoisobutyric acid
  SITE (47)..(56): Where the glutamic acid residues at positions 47-56 have D-chirality
AVSEHQLLHDKGKSIQDLRRRELLEKLLXKLHTAEIRATSEVSPNSEEEEEEEEEE

SEQ ID NO: 2 - protein, 66 aa, Artificial Sequence
  a compound for targeting and treating a spinal fusion site that has a payload comprising abaloparatide
  SITE (29)..(29): Where “X” at position 29 is alpha- aminoisobutyric acid
  SITE (47)..(66): Where “X” at position 29 is alpha- aminoisobutyric acid have D-chirality
AVSEHQLLHDKGKSIQDLRRRELLEKLLXKLHTAEIRATSEVSPNSEEEEEEEEEEEEEEEEEEEE

(19) United States
(12) Patent Application Publication
(10) Pub. No.: US 2023/0390363 A1
(43) Pub. Date: Dec. 7, 2023

(54) Compounds, Compositions and Methods of Use to Treat Spinal Fusions

(71) Applicant: PURDUE RESEARCH FOUNDATION, Suite 1594, IN (US)

(72) Inventors: Philip Stewart LOW, West Lafayette, IN (US); Stewart A. LOW, West Lafayette, IN (US); Jeffrey Jay Howard NIELSEN, West Lafayette, IN (US)

(21) Appl. No.: 18/033,658

(22) PCT Filed: Aug. 26, 2021
(86) PCT No.: PCT/US21/47827

§ 371 Date: Apr. 25, 2023

Related U.S. Application Data
(60) Provisional application No. 63/193,753, filed on May 27, 2021.
(60) Provisional application No. 63/105,678, filed on Oct. 26, 2020.

(51) Int. Cl. A61K 38/29; A61K 47/64; A61K 49/00; A61P 19/08

(52) U.S. Cl. CPC A61K 38/29; A61K 47/64; A61K 49/0056; A61P 19/08

(57) ABSTRACT

Osteotropic ligand-bone anabolic agent compounds and related compositions and methods of use to treat spinal fusion.

### PRIORITY

This patent application is related to and claims the priority benefit of: (a) U.S. Provisional Patent Application No. 63/105,678, filed Oct. 26, 2020, and (b) U.S. Provisional Patent Application No. 63/193,753, filed May 27, 2021. The contents of each of the aforementioned priority applications are hereby incorporated by reference in their entireties.

### STATEMENT OF GOVERNMENT SUPPORT

This invention was made with government support under DE 028713 awarded by the National Institutes of Health. The government has certain rights in the invention.

### TECHNICAL FIELD

The present disclosure relates to osteotropic ligands, bone anabolic agents, conjugates comprising both, compositions comprising the same, and methods of use to treat spinal fusions.

### BRIEF DESCRIPTION OF THE SEQUENCE LISTING

The sequences described herein are set forth in the Figures and also provided in computer-readable form submitted herewith and incorporated herein by reference. The information recorded in computer readable form is identical to the written Sequence Listing provided herein, pursuant to 37 C.F.R. § 1.821(f).

### BACKGROUND

Over 500,000 spinal fusions occur every year in the United States. Spinal fusion is surgery to connect (e.g., permanently) two or more vertebrae in a spine, eliminating motion therebetween. In some instances, they are performed to treat back pain, malformities, and/or vertebral disk and intervertebral disk degeneration. Spinal fusion surgeries can cost up to US$120,000.

Spinal fusion involves techniques designed to mimic the normal healing process of broken bones. As such, a large amount of a therapeutic agent (e.g., growth factor, anti-inflammatory agents, and/or synthetic substances) may be administered to a patient at the time of or after (e.g., spinal fusion) surgery. This can lead to undesirable side effects, such as, for example, ectopic mineralization, which can be caused by leakage of the therapeutic agent into other tissues.

Provided herein are compounds, compositions and methods for treating or improving healing of spinal fusions (e.g., by targeting delivery of anabolic agents to the site of spinal fusion). This and other objects and advantages, as well as inventive features, will be apparent from the description provided herein.

### SUMMARY

In some embodiments, provided herein is a method for treating a bone-healing event (e.g., spinal fusion) of an individual (e.g., in need thereof) comprising administering (e.g., a therapeutically effective amount of) a compound or pharmaceutically acceptable salt thereof provided herein, such as, for example, a compound or pharmaceutically acceptable salt thereof that comprises a bone targeting agent (e.g., an osteotropic ligand) and/or an anabolic agent (e.g., a bone anabolic agent).

In some embodiments, provided herein is a compound having a structure of Formula (I):

X-Y-Z.

In some embodiments, the compound having the structure of Formula (I) is a pharmaceutically acceptable salt thereof.

In some embodiments, X is a bone anabolic agent. The bone anabolic agent can be any suitable bone anabolic agent. The bone anabolic agent can be a parathyroid hormone (PTH), a PTH-related protein (PTHrP), a derivative of either of the foregoing having bone anabolic activity, or a fragment of any of the foregoing having bone anabolic activity. The bone anabolic agent can be abaloparatide, a derivative thereof having bone anabolic activity, or a fragment thereof having bone anabolic activity. In some embodiments, the bone anabolic agent can be preptin, integrin 5 beta 1 (ITGA), dasatinib, or a derivative or fragment of any of the foregoing having bone anabolic activity. In some embodiments, X is abaloparatide, ITGA, dasatinib, PTH, PTHrP, or a derivative or fragment of any one of the foregoing having bone anabolic activity; Y is a non-releasable oligopeptide linker; and Z is DE20. In some embodiments, X is abaloparatide or a derivative or fragment thereof, Y is a releasable oligopeptide linker comprising at least one protease-specific amide bond, and Z is DE20.

In some embodiments, Y is absent or a linker (e.g., a releasable linker or a non-releasable linker). Y, when present, can be a non-releasable linker, such as a non-releasable linker containing at least one carbon-carbon bond and/or at least one amide bond. Alternatively, Y, when present, can be a releasable linker, such as a releasable linker containing at least one disulfide bond, at least one ester, at least one protease-specific amide bond, or a combination of the foregoing.

In some embodiments, Z is an osteotropic ligand (e.g., an acidic oligopeptide (AOP) (e.g., comprising at least 4 amino acid residues (e.g., 4 to 20 amino acid residues))). The amino acid residues can be glutamic acid amino acid residues, aspartic acid amino acid residues, or a mixture thereof. The amino acid residues can have D chirality.

In some embodiments, Z is a linear chain of amino acid residues. In some embodiments, Z is an AOP (e.g., comprising at least 4 glutamic acid amino acid residues or 4 aspartic acid amino acid residues, or both). In some embodiments, Z is a hydroxyapatite binding molecule other than AOP including, for example, a bisphosphonate, a ranolate, and/or a tetracycline.

In some embodiments, Z comprises at least 4 amino acid residues (e.g., 4 or more, 10 or more, 20 or more, 30 or more, 50 or more, 75 or more, or 100 or more). In some embodiments, Z comprises 4 to 75 acidic amino acid residues (e.g., D-glutamic acid amino acid residues). In some embodiments, Z comprises at most 100 amino acid residues (e.g., 100 or less, 75 or less, 50 or less, 30 or less, 20 or less, 10 or less, or 4 or less). In some embodiments, Z comprises not less than 4 and not more than 35 amino acids. In some embodiments, Z comprises not less than 4 and not more than 20 amino acids. In some embodiments, Z comprises not less than 6 and not more than 30 amino acids. In some embodiments, Z comprises not less than 8 and not more than 30 amino acids. In some embodiments, Z comprises not less than 8 and not more than 20 amino acids. In some embodiments, Z comprises glutamic acid amino acid residues. In some embodiments, Z comprises D-glutamic acid amino acid residues.

In some embodiments, Z comprises 4 to 75 D-glutamic acid amino acid residues. In some embodiments, Z comprises 8 to 30 D-glutamic acid amino acid residues. In some embodiments, Z comprises 8 to 20 D-glutamic acid amino acid residues.

In some embodiments, the AOP comprises from about 4 to about 20 amino acid residues (such as 4 to about 20 or about 4 to 20) or more amino acid residues, such as 4, 5, 6, 7, 8, 9, 10, 11, 12, 13, 14, 15, 16, 17, 18, 19 or 20. In various embodiments, the AOP comprises about 20 amino acid residues, such as 20 amino acid residues.

In some embodiments, Z comprises at least 4 (e.g., D-) glutamic acid amino acid residues (e.g., 4 to 20 D-glutamic acid amino acid residues) and/or at least 4 (e.g., D-) aspartic acid amino acid residues (e.g., 4 to 20 D-aspartic acid amino acid residues).

In some embodiments, the amino acid is aspartic acid (represented by the letter D), glutamic acid (represented by the letter E), or a mixture thereof. The amino acid residues can have D chirality, L chirality, or a mixture thereof. In some embodiments, the amino acid residue has D chirality. In some embodiments, the amino acid residue has L chirality. In some embodiments, Z comprises at least 4 (e.g., acidic) amino acid residues (e.g., having the same chirality (e.g., D- or L-amino acid residues)). In some embodiments, each of the at least 4 (e.g., acidic) amino acid residue has D chirality. In some embodiments, the aspartic acid is D-aspartic acid or L-aspartic acid. In some embodiments, the glutamic acid is D-glutamic acid or L-glutamic acid. In some embodiments, Z comprises not less than 4 and not more than 20 D-glutamic acid residues or L-glutamic acid residues. In some embodiments, Z comprises not less than 4 and not more than 20 D-aspartic acid residues or L-aspartic acid residues.

In some embodiments, Z comprises at least 4 (e.g., D-) glutamic acid amino acid residues (e.g., 4 to 20 D-glutamic acid amino acid residues) and/or at least 4 (e.g., D-) aspartic acid amino acid residues (e.g., 4 to 20 D-aspartic acid amino acid residues).

In some embodiments, Z comprises a mixture of (e.g., D-) glutamic acid amino acid residues and (e.g., D-) aspartic acid amino acid residues.

In some embodiments, Z comprises at least 4 repeating D-glutamic acid amino acid residues (DE4) or more (e.g., 6 repeating repeating D-glutamic acid amino acid residues (DE6) or more, 8 repeating D-glutamic acid amino acid residues (DE8) or more, 10 repeating D-glutamic acid amino acid residues (DE10) or more, 15 repeating D-glutamic acid amino acid residues (DE15) or more, 20 repeating D-glutamic acid amino acid residues (DE20) or more, 25 repeating D-glutamic acid amino acid residues (DE25) or more, 30 repeating D-glutamic acid amino acid residues (DE30) or more, or 35 repeating D-glutamic acid amino acid residues (DE35) or more). In some embodiments, Z is DE10 or DE20.

In some embodiments, X is abaloparatide or a derivative or fragment thereof (e.g., having bone anabolic activity)) and Z is DE20. In some embodiments, X is abaloparatide, ITGA, dasatinib, PTH, PTHrP, or a derivative or fragment of any one of the foregoing having bone anabolic activity, and Z is DE20.

As noted above, in some embodiments, Y is a non-releasable linker. In some embodiments, Y is a non-releasable linker containing at least one carbon-carbon bond. In some embodiments, Y is a non-releasable linker containing at least one amide bond. In some embodiments, Y is a non-releasable linker containing at least one carbon-carbon bond and at least one amide bond.

In some embodiments, Y is a non-releasable linker and comprises one or more amide bond(s). In some embodiments, Y is a non-releasable linker and comprises 1-20 amide bond(s). In some embodiments, Y is a non-releasable linker and comprises 1-10 amide bond(s). In some embodiments, Y is a non-releasable linker and comprises 10-20 amide bond(s). In some embodiments, Y is a non-releasable linker and comprises 1-5 amide bond(s).

In some embodiments, Y is a non-releasable linker and comprises one or more amino acid linker group(s). In some embodiments, Y is a polypeptide. In some embodiments, the polypeptide comprises 1-20 amino acid residue(s). In some embodiments, the polypeptide comprises 1-10 amino acid residue(s). In some embodiments, the polypeptide comprises 10-20 amino acid residue(s). In some embodiments, the polypeptide comprises 1-5 amino acid residue(s).

In some embodiments, Y is a non-releasable linker and comprises one or more ether bond(s) (C—O). In some embodiments, Y is a non-releasable linker and comprises 1-20 ether bond(s) (C—O). In some embodiments, Y is a non-releasable linker and comprises 1-10 ether bond(s) (C—O). In some embodiments, Y is a non-releasable linker and comprises 10-20 ether bond(s) (C—O). In some embodiments, Y is anon-releasable linker and comprises 1-5 ether bond(s) (C—O).

In some embodiments, Y is a non-releasable linker and comprises one or more polyethylene glycol (PEG) linker group. In some embodiments, Y is a PEG.

In some embodiments, Y is a non-releasable linker and comprises one or more thioether bond(s) (C—S). In some embodiments, Y is a non-releasable linker and comprises 1 thioether bond (C—S). In some embodiments, Y is a non-releasable linker and comprises 1-20 thioether bond(s) (C—S). In some embodiments, Y is a non-releasable linker and comprises 1-10 thioether bond(s) (C—S). In some embodiments, Y is a non-releasable linker and comprises 10-20 thioether bond(s) (C—S). In some embodiments, Y is a non-releasable linker and comprises 1-5 thioether bond(s) (C—S).

In some embodiments, Y is a releasable linker. In some embodiments, Y is a releasable linker containing at least one disulfide (S—S). In some embodiments, Y is a releasable linker containing at least one ester (e.g., O(C═O)). In some embodiments, Y is a releasable linker containing at least one (e.g., protease-specific) amide bond.

In some embodiments, Y is a releasable linker and comprises one or more amide bond(s). In some embodiments, Y is a releasable linker and comprises 1-20 amide bond(s). In some embodiments, Y is a releasable linker and comprises 1-10 amide bond(s). In some embodiments, Y is a releasable linker and comprises 10-20 amide bond(s). In some embodiments, Y is a releasable linker and comprises 1-5 amide bond(s).

In some embodiments, Y is a releasable linker and comprises one or more amino acid linker group(s). In some embodiments, Y is a polypeptide. In some embodiments, the polypeptide comprises 1-20 amino acid residue(s). In some embodiments, the polypeptide comprises 1-10 amino acid residue(s). In some embodiments, the polypeptide comprises 10-20 amino acid residue(s). In some embodiments, the polypeptide comprises 1-5 amino acid residue(s).

In some embodiments, Y is a releasable linker and comprises one or more ether bond(s) (C—O). In some embodiments, Y is a releasable linker and comprises 1-20 ether bond(s) (C—O). In some embodiments, Y is a releasable linker and comprises 1-10 ether bond(s) (C—O). In some embodiments, Y is a releasable linker and comprises 10-20 ether bond(s) (C—O). In some embodiments, Y is a releasable linker and comprises 1-5 ether bond(s) (C—O).

In some embodiments, Y is a releasable linker and comprises one or more PEG linker group(s).

In some embodiments, X is abaloparatide (e.g., or a derivative or fragment thereof (e.g., having bone anabolic activity)), Y is a releasable oligopeptide linker comprising at least one protease-specific amide bond, and Z is DE20.

In some embodiments, X is abaloparatide (e.g., or a derivative or fragment thereof (e.g., having bone anabolic activity)), Y is a non-releasable oligopeptide linker, and Z is DE10. In some embodiments, the compound is SEQ ID NO: 1.

In some embodiments, X is abaloparatide or a derivative or fragment thereof (e.g., having bone anabolic activity)), Y is a non-releasable oligopeptide linker, and Z is DE20. In some embodiments, the compound is SEQ ID NO: 2.

In some embodiments, X is a peptide (e.g., a polypeptide). In some embodiments, a compound having the structure of Formula (I) is a (poly)peptide.

In some embodiments, provided herein is a (poly)peptide having bone anabolic activity (e.g., abaloparatide or a derivative or fragment thereof). In some embodiments, provided herein is a substantially pure (poly)peptide having bone anabolic activity (e.g., abaloparatide), wherein the (poly)peptide comprises an amino acid sequence having at least 75%, at least 85%, at least 95% amino acid sequence identity with an amino acid sequence set forth in SEQ ID NO: 1 or SEQ ID NO: 2. In some embodiments, SEQ ID NO: 1 has bone anabolic activity (and, for example, bone targeting activity). In some embodiments, SEQ ID NO: 2 has bone anabolic activity (and, for example, bone targeting activity). In other embodiments, the (poly)peptide comprises an amino acid sequence having at least 75% sequence identity (e.g., at least 75% sequence identity or more, at least 85% sequence identity or more, at least 90% sequence identity or more, or at least 95% sequence identity or more) with the amino acid sequence set forth in SEQ ID NO: 1. In other embodiments, the (poly)peptide comprises an amino acid sequence having at least 75% sequence identity (e.g., at least 75% sequence identity or more, at least 85% sequence identity or more, at least 90% sequence identity or more, or at least 95% sequence identity or more) with the amino acid sequence set forth in SEQ ID NO: 2. In certain embodiments, the poly(peptide) comprises PTH, a PTHrP, abaloparatide (Abalo), or a derivative or fragment of any of the foregoing, or an amino acid sequence having at least 75% sequence identity (e.g., at least 75% sequence identity or more, at least 85% sequence identity or more, at least 90% sequence identity or more, or at least 95% sequence identity or more) with a nucleotide sequence thereof.

In another embodiment, the (poly)peptide is an amino acid sequence having at least 75% sequence identity or more, at least 85% sequence identity or more, at least 90% sequence identity or more, or at least 95% sequence identity or more to an amino acid sequence shown in FIG. 1. In another embodiment, the (poly)peptide is an amino acid sequence shown in FIG. 1.

In some embodiments, a compound provided herein comprises a payload. In some embodiments, the payload comprises abaloparatide (Abalo) or a derivative or fragment thereof (e.g., having bone anabolic activity)) (e.g., SEQ. ID. NO: 2). In some embodiments, the payload comprises a linker provided herein. In some embodiments, the payload and a linker provided herein, wherein the payload comprises abaloparatide (Abalo) or a derivative or fragment thereof (e.g., having bone anabolic activity)) (e.g., SEQ. ID. NO: 2).

Provided in some embodiments herein is a conjugate of Formula (I) having the following structure: X-Y-Z.

In another embodiment, the linker is an amino acid sequence having at least 75% sequence identity or more, at least 85% sequence identity or more, at least 90% sequence identity or more, or at least 95% sequence identity or more to an amino acid sequence shown in FIG. 1. In another embodiment, the linker comprises a portion of an amino acid sequence shown in FIG. 1.

In some embodiments, Z is an osteotropic ligand, which can be an AOP comprising at least 11 to 100 amino acid residues.

The amino acid residues can be glutamic acid, aspartic acid, or a mixture thereof. The amino acid residues can have D chirality. The AOP can be a linear chain of amino acid residues. When Y is present, Y can be a non-releasable linker containing at least one carbon-carbon bond and/or at least one amide bond. Alternatively, when Y is present, Y can be a releasable linker containing at least one disulfide, ester, and/or protease-specific amide bond.

In some embodiments, the (poly)peptide is a pharmaceutically acceptable salt of any compound provided herein (e.g., a compound having a structure of Formula (I), SEQ ID NO: 1, or SEQ ID NO: 2).

In some embodiments, provided herein is a pharmaceutical composition comprising any compound provided herein (e.g., a compound having a structure of Formula (I), SEQ ID NO: 1, or SEQ ID NO: 2), or a pharmaceutically acceptable salt thereof (e.g., and at least one pharmaceutically acceptable carrier or excipient).

In some embodiments, the compound provided herein (e.g., the compound having the structure of Formula (I), SEQ ID NO: 1 or SEQ ID NO: 2) is administered (e.g., subcutaneously) to an individual (e.g., a patient or an individual in need thereof).

In some embodiments, provided herein is a method of treating a spinal fusion (e.g., of an individual (e.g., a patient or an individual in need thereof)). In some embodiments, the method comprises administering (e.g., subcutaneously) a therapeutically effective amount of any compound or pharmaceutical composition provided herein (e.g., a compound having a structure of Formula (I), SEQ ID NO: 1 or SEQ ID NO: 2) to the individual (e.g., a patient or an individual in need thereof). In some embodiments, administering (e.g., subcutaneously) a therapeutically effective amount of any compound provided herein (e.g., a compound having a structure of Formula (I), SEQ ID NO: 1 or SEQ ID NO: 2) to the individual (e.g., a patient or an individual in need thereof) treats the spinal fusion or improves the healing of the spinal fusion of the individual (e.g., a patient or an individual in need thereof).

In some embodiments, provided herein is a method for localizing a compound provided herein (e.g., a compound having a structure of Formula (I), SEQ ID NO: 1 or SEQ ID NO: 2) to a spinal fusion of an individual in need thereof (e.g., suffering from or having undergone a spinal fusion) comprising administering (e.g., subcutaneously) an amount of any compound provided herein (e.g., a compound having a structure of Formula (I), SEQ ID NO: 1 or SEQ ID NO: 2) to the individual (e.g., a patient or an individual in need thereof). In some embodiments, administering (e.g., subcutaneously) the amount of any compound provided herein (e.g., a compound having a structure of Formula (I), SEQ ID NO: 1 or SEQ ID NO: 2) to the individual (e.g., a patient or an individual in need thereof) localizes the compound provided herein to a spinal fusion (e.g., in the individual).

In some embodiments, X is abaloparatide or a derivative or fragment thereof (e.g., having bone anabolic activity)), Y is a releasable oligopeptide linker comprising at least one protease-specific amide bond, and Z is DE20.

In some embodiments, X is abaloparatide or a derivative or fragment thereof (e.g., having bone anabolic activity)), Y is a non-releasable oligopeptide linker, and Z is DE10. In some embodiments, the compound is SEQ ID NO: 1.

In some embodiments, X is abaloparatide or a derivative or fragment thereof (e.g., having bone anabolic activity)), Y is a non-releasable oligopeptide linker, and Z is DE20. In some embodiments, the compound is SEQ ID NO: 2.

A method of treating a spinal fusion in a patient is provided. The method comprises administering (e.g., subcutaneously) to the patient an effective amount of either (i) a conjugate of formula X-Y-Z, wherein:
X is a bone anabolic agent,
Y, when present, is a linker, which can be either releasable or non-releasable, and
Z is an osteotropic ligand (e.g., an AOP comprising at least 4 amino acid residues to 20 amino acid residues), or (ii) a pharmaceutical composition comprising the conjugate or pharmaceutically acceptable salt thereof of subpart (i) and a pharmaceutically acceptable carrier.

In some embodiments, a compound provided herein is administered (e.g., to an individual in need thereof) subcutaneously. In some embodiments, a compound or pharmaceutical composition thereof (or a pharmaceutically acceptable salt thereof) can be administered directly to a spinal fusion site.

In some embodiments, a therapeutically effective amount of any compound or pharmaceutical composition provided herein is administered daily, weekly, bi-weekly, or monthly (e.g., for a period of time, such as, for example, one week, one month, one year, or longer). In some embodiments, a therapeutically effective amount of any compound or pharmaceutical composition provided herein is administered once or twice weekly.

Also provided is a kit for treating a spinal fusion in a patient and/or targeting a therapeutic or diagnostic agent to the spinal fusion. The kit comprises (a) (i) a compound or conjugate of Formula (I) having a structure of X-Y-Z or a pharmaceutically acceptable salt thereof (e.g., also comprising a pharmaceutically acceptable carrier) as provided herein. In some embodiments, for example, X is a therapeutic agent for treating a spinal fusion in the patient (e.g., a bone anabolic agent) or a diagnostic agent for identifying a spinal fusion in the patient, Y, when present (e.g., Y can be absent), is a linker, which can be either releasable or non-releasable, and Z is an osteotropic ligand (e.g., an acidic oligopeptide (AOP) comprising at least 4 amino acid residues to 20 amino acid residues and/or other hydroxyapatite binding molecule including, for example, a bisphosphonate, a ranolate, and/or a tetracycline); and (b) a collagen sponge, a mineralized collagen, or a bone graft. The therapeutic agent can be a bone anabolic agent. In some embodiments, the therapeutic agent can be a PTH, a PTHrP, a derivative of either of the foregoing having bone anabolic activity, or a fragment of any of the foregoing having bone anabolic activity. The bone anabolic agent can be abaloparatide, a derivative thereof having bone anabolic activity, or a fragment thereof having bone anabolic activity. In some embodiments, the therapeutic agent (e.g., a bone anabolic agent) can be selected from the group consisting of abaloparatide, ITGA, dasatinib, PTH, PTHrP, and a derivative or fragment of any of the foregoing having bone anabolic activity.

In some embodiments of the kits hereof, where X is a therapeutic agent comprising a bone anabolic agent, X is a PTH or a PTHrP or a derivative or fragment thereof (e.g., having bone anabolic activity). In some embodiments, the bone anabolic agent is a PTH or a derivative or fragment thereof. In some embodiments, the bone anabolic agent is a PTHrP or a derivative or fragment thereof. In some embodiments, the bone anabolic agent is a (e.g., synthetically) modified PTH or a derivative or fragment thereof. In some embodiments, the bone anabolic agent is a (e.g., synthetically) modified PTHrP or a derivative or fragment thereof. In some embodiments, the bone anabolic agent is abaloparatide or a derivative or fragment thereof (e.g., having bone anabolic activity). In some embodiments, the bone anabolic agent is abaloparatide. In some embodiments, the bone anabolic agent is a (e.g., synthetically) modified abaloparatide.

In some embodiments, where X comprises a therapeutic agent (e.g., a bone anabolic agent), administration of a therapeutically effective amount of the compound having a structure of Formula (I) to the patient treats the spinal fusion.

When X comprises a diagnostic agent, the diagnostic agent can be a fluorescent dye or any other imaging agent suitable for identifying (e.g., visually) a spinal fusion present within the patient. In some embodiments where X comprises a diagnostic agent, administration of the compound having a structure of Formula (I) to the patient identifies a spinal fusion if present.

In some embodiments, Z is a linear chain of amino acid residues. In some embodiments, Z is an AOP (e.g., comprising at least 4 glutamic acid amino acid residues or 4 aspartic acid amino acid residues).

In some embodiments, Z comprises at least 4 amino acid residues (e.g., 4 or more, 10 or more, 20 or more, 30 or more, 50 or more, 75 or more, or 100 or more). In some embodiments, Z comprises at most 100 amino acid residues (e.g., 100 or less, 75 or less, 50 or less, 30 or less, 20 or less, 10 or less, or 4 or less). In some embodiments, Z comprises not less than 4 and not more than 35 amino acids. In some embodiments, Z comprises not less than 4 and not more than 20 amino acids. In some embodiments, Z comprises not less than 6 and not more than 30 amino acids. In some embodiments, Z comprises not less than 8 and not more than 30 amino acids. In some embodiments, Z comprises not less than 8 and not more than 20 amino acids.

In some embodiments, the AOP the comprises from about 4 to about 20 amino acid residues (such as 4 to about 20 or about 4 to 20) or more amino acid residues, such as 4, 5, 6, 7, 8, 9, 10, 11, 12, 13, 14, 15, 16, 17, 18, 19 or 20. In various embodiments, the AOP comprises about 20 amino acid residues, such as 20 amino acid residues.

In some embodiments, the amino acid is aspartic acid (represented by the letter D), glutamic acid (represented by the letter E), or a mixture thereof. The amino acid residues can have D chirality, L chirality, or a mixture thereof. In some embodiments, the amino acid residue has D chirality. In some embodiments, the amino acid residue has L chirality. In some embodiments, Z comprises at least 4 (e.g., acidic) amino acid residues having the same chirality (e.g., D- or L-amino acid residues). In some embodiments, each of the at least 4 (e.g., acidic) amino acid residue has D chirality. In some embodiments, the aspartic acid is D-aspartic acid or L-aspartic acid. In some embodiments, the glutamic acid is D-glutamic acid or L-glutamic acid. In some embodiments, Z comprises not less than 4 and not more than 20 D-glutamic acid residues or L-glutamic acid residues. In some embodiments, Z comprises not less than 4 and not more than 20 D-aspartic acid residues or L-aspartic acid residues.

In some embodiments, Z comprises at least 4 (e.g., D-) glutamic acid amino acid residues (e.g., 4 to 20 D-glutamic acid amino acid residues) and/or at least 4 (e.g., D-) aspartic acid amino acid residues (e.g., 4 to 20 D-aspartic acid amino acid residues).

In some embodiments, Z comprises a mixture of (e.g., D-) glutamic acid amino acid residues and (e.g., D-) aspartic acid amino acid residues.

In some embodiments, Z comprises at least DE4 or more (e.g., DE6 or more, DE8 or more, DE10 or more, DE15 or more, or DE20 or more, DE25 or more, DE30 or more, or DE35 or more). In some embodiments, Z comprises at least 10 repeating D-glutamic acid amino acid residues (e.g., DE10 or more, DE15 or more, or DE20 or more, DE25 or more, DE30 or more, or DE35 or more). In some embodiments, X is abaloparatide or a derivative or fragment thereof (e.g., having bone anabolic activity) and Z is DE20.

In some embodiments, Y is a non-releasable linker. In some embodiments, Y is a non-releasable linker containing at least one carbon-carbon bond. In some embodiments, Y is a non-releasable linker containing at least one amide bond. In some embodiments, Y is a non-releasable linker containing at least one carbon-carbon bond and at least one amide bond.

In some embodiments, Y is a releasable linker. In some embodiments, Y is a releasable linker containing at least one disulfide (S—S). In some embodiments, Y is a releasable linker containing at least one ester (e.g., O(C═O)). In some embodiments, Y is a releasable linker containing at least one (e.g., protease-specific) amide bond.

In some embodiments, Y is a linker described elsewhere herein (e.g., hereinabove).

In some embodiments, Z is an osteotropic ligand described elsewhere herein (e.g., hereinabove).

In some embodiments, X is abaloparatide or a derivative or fragment thereof (e.g., having bone anabolic activity), Y is a non-releasable oligopeptide linker, and Z is DE20.

In some embodiments, X is abaloparatide or a derivative or fragment thereof (e.g., having bone anabolic activity), Y is a releasable oligopeptide linker comprising at least one protease-specific amide bond, and Z is DE20.

In some embodiments, the compound is an imaging agent (e.g., a dye).

In some embodiments, the compound is any of the compounds described herein.

In some embodiments, the compound is SEQ ID NO: 1. In some embodiments, the compound is SEQ ID NO: 2.

In some embodiments, provided herein is a pharmaceutical composition comprising any compound provided herein (e.g., a compound having a structure of Formula (I), SEQ ID NO: 1 or SEQ ID NO: 2), or a pharmaceutically acceptable salt thereof (e.g., and at least one pharmaceutically acceptable carrier or excipient). In some embodiments, the pharmaceutical composition is formulated for subcutaneous administration to the patient. In some embodiments of the pharmaceutical composition, X is a therapeutic agent that is a bone anabolic agent. In some embodiments, X is a diagnostic agent that is an imaging agent (e.g., a fluorescent dye).

Methods are also provided for localizing a therapeutic agent or a diagnostic agent to a spinal fusion site in a patient. In some embodiment, such methods comprise administering to the patient a therapeutically effective amount of any of the compounds or pharmaceutical compositions described herein.

Further embodiments and the full scope of applicability of the present disclosure will become apparent from the Detailed Description. However, it should be understood that the Detailed Description and specific examples are given by way of illustration only. Various changes and modifications within the spirit and scope of the present disclosure will become apparent to those skilled in the art.

### BRIEF DESCRIPTION OF THE FIGURES

FIG. 1 shows the amino acid sequences of SEQ ID NO: 1 and SEQ ID NO: 2.

FIG. 2 shows the biodistribution of a compound provided herein post-injection with a collagen scaffold two weeks post-surgery.

FIG. 3 shows the biodistribution of a compound provided herein post-injection with a mineralized collagen scaffold two weeks post-surgery.

FIG. 4 shows the biodistribution of a compound provided herein post-injection with bone graft scaffold two weeks post surgery.

FIG. 5 shows the biodistribution of a compound provided herein post-injection eight weeks post-surgery.

FIG. 6 shows a three-dimensional reconstruction of micro-CT scans of a spinal fusion of a rat treated with a collagen matrix and a compound provided herein for eight weeks, where Panel A is a left medial view, Panel B is a dorsal view and Panel C is a right medial view.

FIG. 7 shows a three-dimensional reconstruction of micro-CT scans of a spinal fusion of a rat treated with a collagen matrix soaked with rhBMP-2 for eight weeks, where Panel A is a left medial view, Panel B is a dorsal view, and Panel C is a right medial view.

FIG. 8 shows a three-dimensional reconstruction of micro-CT scans of a spinal fusion of a rat treated with a collagen matrix and saline for eight weeks, where Panel A is a left medial view, Panel B is a dorsal view, and Panel Cis a right medial view.

FIG. 9 shows a three-dimensional reconstruction of micro-CT scans of a spinal fusion of a rat treated with a bone graft matrix and rhBMP-2 (10 μg) for eight weeks, where Panel A is a left medial view, Panel Bis a dorsal view, and Panel C is a right medial view.

FIG. 10 shows a three-dimensional reconstruction of micro-CT scans of a spinal fusion of a rat treated with a bone graft matrix and a compound provided herein for eight weeks, where Panel A is a left medial view, Panel B is a dorsal view, and Panel C is a right medial view.

FIG. 11 shows a three-dimensional reconstruction of micro-CT scans of a spinal fusion of a rat treated with a bone graft matrix and saline for eight weeks, where Panel A is a left medial view, Panel B is a dorsal view, and Panel C is a right medial view.

FIG. 12 shows a three-dimensional reconstruction of micro-CT scans of a spinal fusion of a rat treated with a mineralized collagen scaffold and saline one week post-implantation.

FIG. 13 shows a three-dimensional reconstruction of micro-CT scans of spinal fusions of rats treated with a mineralized collagen scaffold and different treatments after eight weeks of treatment. Panels A and B show three-dimensional reconstructions of micro-CT scans for spinal fusion treated with mineralized collagen and BMP-2 after eight weeks. Panels C and D show three-dimensional reconstructions of micro-CT scans for spinal fusion treated with mineralized collagen and a compound provided herein after eight weeks. Panels E and F show three-dimensional reconstructions of micro-CT scans for spinal fusion treated with mineralized collagen and saline after eight weeks.

FIG. 14 shows a three-dimensional reconstruction of micro-CT scans of spinal fusions of rats treated with a mineralized collagen scaffold and different treatments after five weeks of treatment. Panel A shows a three-dimensional reconstructions of micro-CT scans for spinal fusion treated with mineralized collagen and a compound provided herein after five weeks. Panel B shows a three-dimensional reconstruction of micro-CT scans for spinal fusion treated with mineralized collagen and rhBMP-2 after five weeks. Panel C shows three-dimensional reconstruction of micro-CT scans for spinal fusion treated with mineralized collagen and saline after five weeks of treatment.

FIG. 15 shows a graph of weeks vs. total fusion score for BMP and a compound provided herein.

FIG. 16 is a graph of weeks vs. total fusion score for saline, BMP and a compound provided herein.

FIG. 17 is a graph of weeks vs. bone mineral density (BMD) (HA mg/cm 3) for a compound provided herein and saline.

FIG. 18 shows the total area (a graph of week vs. bone volume (BV) (mm 3) of mineralized tissue in the bone bridges formed by spinal fusions treated with a compound provided herein or saline.

### DETAILED DESCRIPTION

The present disclosure relates to the preparation and use of compounds and compositions that treat and/or facilitate treatment of spinal fusions. In some embodiments, the compounds, compositions, and methods leverage strategies to (e.g., selectively) localize the therapeutic agents to a spinal fusion site. For example, the compounds, compositions, and methods provided may comprise an osteotropic ligand that, localizes the compounds and compositions to a spinal fusion site in a patient. In some embodiments, the compounds and compositions are formulated to exhibit increased retention time (such as due to increased resistance to degradation, for example) such that the frequency at which the compound or composition is readministered to maintain a therapeutically effective concentration at the targeted site (e.g., a spinal fusion) is reduced. Kits are also provided for treating a spinal fusion in a patient and/or targeting a therapeutic or diagnostics agent to a spinal fusion in the patient.

The compounds, compositions, and methods hereof allow for significant advantages over conventional spinal fusion therapies. In some embodiments, the noninvasive targeted methods of drug administration for spinal fusions allow for repeated drug administration specifically to spinal fusions, thus, allowing for therapeutic levels of the drug to stimulate the repair of the spinal fusion for a longer period of time than what is available with conventional methodologies. This increased duration of therapeutic levels of the compounds and/or compositions can increase the efficacy and effect of anabolics relative to drugs administered locally (e.g., as a bolus) and can ultimately lead to faster repair of the spinal fusion. The targeted nature of the compounds, compositions, and methods also reduces systemic side effects and side effects induced by compound leakage from the implantation site (for example, as is often the case with a therapeutic like in bone morphogenetic protein-2 (BMP2)). Additionally, the compounds, compositions, and methods are non-invasive which allows for temporal control over drug administration. This can result in more control for physicians with respect to when and for how long the compounds and compositions are administered such that they can easily adjust for different phases of bone healing and account for patient to patient variability in healing responses.

The requirement that approved osteogenic drugs be topically applied during surgery (e.g., spinal fusion surgery) limits their utility. Moreover, the metabolic turnover of approved bone anabolic agents is relatively fast, which restricts the duration of their therapeutic benefits to a brief window following topical administration during surgery. However, application of a large amount of the therapeutic agent at the time of surgery can lead to undesirable side effects such as ectopic bone growth. In addition to ectopic mineralization due to leakage of locally applied anabolic drugs into surrounding tissues, systemic administration can stimulate unwanted anabolic processes in healthy tissues, such as nerves, muscles, and vasculature. Indeed, hypercalcemia, hypertension, immunosuppression, and even cancer are among the concerns surrounding systemic administration of bone anabolic drugs.

A possible solution is bone targeting. To date, bone targeting has primarily focused on delivering payloads to orthopedic pathologies not related to fractures, such as osteoporosis, osteomyelitis, and bone metastases. Most of these treatments are bisphosphonates to deliver compounds selectively to bone. However, when treating bone fractures, it is imperative to deliver compounds selectively to the fracture site to avoid ectopic ossification that can occur when a drug is delivered nonspecifically to all bone. While tetracycline may be moderately selective for fractured over healthy bone, tetracyclines can be toxic to bone, liver and kidney and are thus not an ideal solution.

Several limitations also exist with using bisphosphonates for fracture targeting, including that they inhibit osteoclasts, which are essential for both normal skeletal remodeling and resolving of fracture calluses from woven bone into laminar bone. Another problem with using bisphosphonates as targeting ligands is that they have half-lives of up to 20 years in bone, which, depending on the stability of their therapeutic cargoes, can potentially lead to an undesirably prolonged stimulation of their molecular targets.

Similar targeting can be observed with ranelates. These compounds can be used as targeting molecules for many bone diseases and can be attached to anabolic agents to speed bone growth and healing. However, like bisphosphonates, they have a long bone half-life.

Given the issues with bisphosphonates, ranelates, and polyphosphates (which include, without limitation, cumbersome synthesis and poor solubility), there remains a need for an osteotropic ligand that does not present such disadvantages. Desirably, the osteotropic ligand can deliver an attached peptidic, therapeutic agent or diagnostic agent to a fracture, in particular a fracture callus.

Abaloparatide is an anabolic, 34-amino acid, synthetic analog of parathyroid hormone-related protein (PTHrP). It can help promote bone growth and conserve bone density and can be used to treat osteoporosis. Abaloparatide acts similarly to PTHrP and targets, binds to, and activates the parathyroid hormone 1 (PTH1) receptor (PTH1R).

PTH1R is a G protein-coupled receptor (GPCR) expressed in osteoblasts and bone stromal cells. PTH1R, in turn, activates the cyclic adenosine monophosphate (cAMP) signaling pathway and the bone anabolic signaling pathway, leading to bone growth and increased bone mineral density and volume. The increase in bone mass and strength helps prevent/treat osteoporosis and decrease the risk of fractures.

Compounds

Provided in some embodiments herein are compounds comprising acidic oligopeptides (AOPs) (e.g., 10-mers and 20-mers of acidic amino acids, 10-mers and 20-mers of either aspartic acid or glutamic acid, or various combinations of the foregoing). In some embodiments, AOPs effectively target spinal fusions. In some embodiments, 20-mers are more effective than 10-mers. In some embodiments, AOPs are highly selective compared to bisphosphonates and tetracyclines. In some embodiments, glutamic acid polymers and aspartic acid polymers have similar retention times at the delivery site. In some embodiments, while oligo-aspartic acids have reduced nonspecific retention in the kidneys, the slight increase in retention time observed with oligo-glutamic acid is transient. In some embodiments, both aspartic acid oligopeptides and glutamic acid oligopeptides (e.g., nearly quantitatively) clear from the kidneys after 18 hours. In some embodiments, AOPs target peptides of all chemical classes (e.g., hydrophobic, neutral, cationic, anionic, short oligopeptides, and long polypeptides) which can be particularly beneficial in the context of targeting a spinal fusion site as it allows for the development of other therapeutics from many chemical classes that are targeted to the spinal fusion by AOPs. While many bone anabolic agents are peptidic, their physical properties can vary greatly and, thus, many are not suitable and/or capable of targeting a desired bone site without conjugation to a targeting ligand.

Provided in some embodiments herein are compounds comprising non-natural D enantiomers of AOPs, which can, in some instances, exhibit increased retention time (such as due to increased resistance to degradation) on the fracture surface compared to the respective L enantiomers. This can be due to an increased resistance to degradation as compared to other compounds, for example. In some embodiments, increased retention time impacts the frequency that a therapeutic agent requires re-administration to maintain a therapeutically effective concentration at the site of surgery (e.g., spinal fusion). In some embodiments, increased retention time impacts the amount of a therapeutic agent required to be administered to elicit a targeted response (e.g., a therapeutic response). In some embodiments, linear AOPs are superior to branched AOPs (e.g., due to a reduction in, or the absence of, steric interference).

In some embodiments, targeted delivery of anabolic agents provides localization of therapeutic agents to bone fracture (e.g., via injection, such as, for example, subcutaneous injection, for example, at a distal site). In some embodiments, a compound provided herein is administered repeatedly to a patient (e.g., in need thereof). In some embodiments, a compound provided herein is administered at a relatively low dose to a patient (e.g., in need thereof). In some embodiments, a compound provided herein is administered at a safe dose to a patient (e.g., in need thereof). In some embodiments, a compound provided herein is administered at a therapeutic dose to a patient (e.g., in need thereof). In some embodiments, targeted delivery minimizes (e.g., if not eliminates) drift of an anabolic agent (e.g., into other tissues and unwanted mineralization). In some embodiments, bone growth in the region is stimulated for a relatively long time (e.g., to achieve relatively fast results (e.g., so that patients can regain their post-surgery mobility more quickly as compared to non-targeted delivery approaches)).

In some embodiments, provided herein is a compound having a structure of Formula (I):

X-Y-Z Formula (I)

or a pharmaceutically acceptable salt thereof, wherein:
X is a bone anabolic agent;
Y is absent or, when present, is a linker, which can be either releasable or non-releasable; and
Z is an osteotropic ligand (e.g., an AOP comprising at least 4 amino acid residues to 20 amino acid residues).

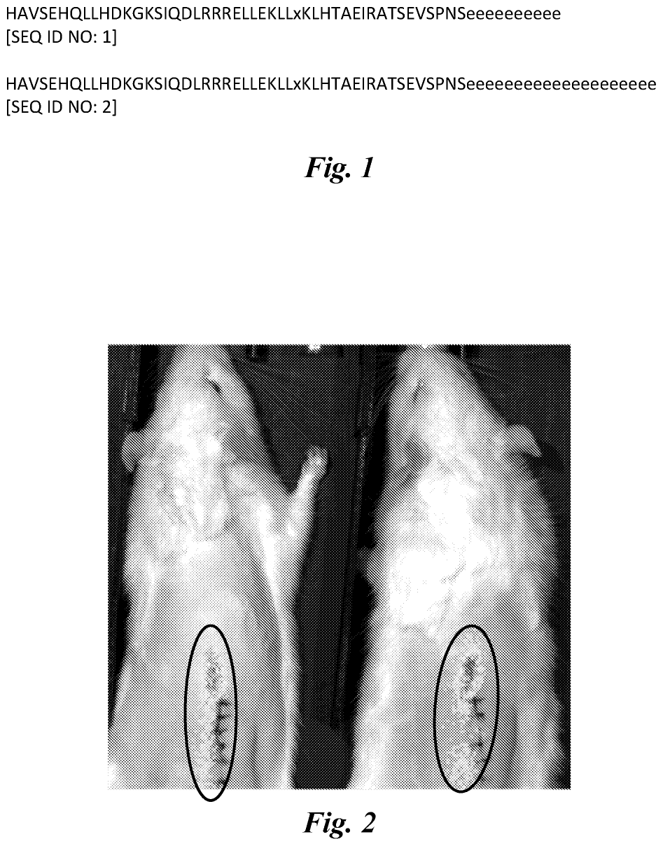

In some embodiments, the compound is any compound, or a pharmaceutically acceptable salt thereof, provided herein (e.g., a compound having a structure of Formula (I), SEQ ID NO: 1, or SEQ ID NO: 2 (see FIG. 1)).

In some embodiments, targeted delivery of agents (e.g., therapeutic agents such as anabolic agents or diagnostic agents such as imaging agents (e.g., fluorescent dye)) (e.g., via the osteotropic ligand (Z)) provides localization of the agents to spinal fusions (e.g., via injection, such as, for example, subcutaneous injection, for example, at a distal site). In some embodiments, a compound provided herein is administered repeatedly to a patient (e.g., in need thereof). In some embodiments, a compound provided herein is administered at a relatively low dose to a patient (e.g., in need thereof). In some embodiments, a compound provided herein is administered at a safe dose to a patient (e.g., in need thereof). In some embodiments, a compound provided herein is administered at a therapeutic dose to a patient (e.g., in need thereof). In some embodiments, targeted delivery minimizes (e.g., if not eliminates) drift of an anabolic agent (e.g., into other tissues and unwanted mineralization). In some embodiments, bone growth in the region is stimulated for a relatively long time (e.g., to achieve relatively fast results (e.g., so that patients can regain their post-surgery mobility more quickly as compared to non-targeted delivery approaches)).

Referring back to the compound or pharmaceutically acceptable salt thereof having the structure of Formula (I), Z can be any suitable osteotropic ligand. In some embodiments, the osteotropic ligand has an affinity for bone, for example, hydroxyapatite. In some embodiments, the osteotropic ligand helps direct the compound (or a derivative or fragment thereof) to bone. In some embodiments, the osteotropic ligand has the potential to target the bone anabolic agent to a spinal fusion. In some embodiments, the osteotropic ligand is a ligand with affinity for hydroxyapatite. In some embodiments, the osteotropic ligand is a ranolate, a bisphosphonate (e.g., alendronate), a tri-bisphosphonate, a tetracycline, a polyphosphate, an acidic molecule (such as a molecule with two or more carboxylic acids), a calcium chelator, a metal chelator, or an AOP. In some embodiments, the osteotropic ligand is an AOP.

In some embodiments, Z comprises at least 4 amino acid residues (e.g., 4 or more, 10 or more, 20 or more, 30 or more, 50 or more, 75 or more, or 100 or more). In some embodiments, Z comprises at most 100 amino acid residues (e.g., 100 or less, 75 or less, 50 or less, 30 or less, 20 or less, 10 or less, or 4 or less). In some embodiments, Z comprises not less than 4 and not more than 30 amino acids. In some embodiments, Z comprises not less than 4 and not more than 20 amino acids. In some embodiments, the AOP the comprises from about 4 to about 20 amino acid residues (such as 4 to about 20 or about 4 to 20) or more amino acid residues, such as 4, 5, 6, 7, 8, 9, 10, 11, 12, 13, 14, 15, 16, 17, 18, 19 or 20. In various embodiments, the AOP comprises about 20 amino acid residues, such as 20 amino acid residues. In other embodiments, the AOP can comprise more than 20 amino acid residues, such as 21, 22, 23, 24, 25, 30, 35, 40, 45, 50, 55, 60, 65, 70, 75, 80, 85, 90, 95 or as many as 100 amino acid residues.

In some embodiments, the amino acids can be aspartic acid (represented by the letter D), glutamic acid (represented by the letter E), or a mixture thereof. The amino acid residues can have D chirality, L chirality, or a mixture thereof. In some embodiments, the amino acid residue has D chirality. In some embodiments, the amino acid residue has L chirality. In some embodiments, the aspartic acid is D-aspartic acid or L-aspartic acid. In some embodiments, the glutamic acid is D-glutamic acid or L-glutamic acid. In some embodiments, Z comprises not less than 4 and not more than 20 D-glutamic acid residues or L-glutamic acid residues. In some embodiments, Z comprises not less than 4 and not more than 20 D-aspartic acid residues or L-aspartic acid residues.

In some embodiments, the AOP comprises one or more neutral or basic amino acids (e.g., provided that the AOP functions effectively as an osteotropic ligand). In some embodiments, the AOP comprises one or more synthetic amino acids (e.g., which can be acidic, neutral or basic).

In some embodiments, the AOP is linear (a linear chain) or branched (a branched chain). A linear chain is used in various embodiments. In some embodiments, the AOP can be cyclized.

The osteotropic ligand (Z) can be a single unit, a polymer, a dendrimer, or multiple units. In some embodiments, the osteotropic ligand is a polymer.

In some embodiments, X is a diagnostic agent for identifying a targeted site. In some embodiments, X is any suitable imaging agent such as, for example, a fluorescent dye. Due to the targeting characteristics of the osteotropic ligand of the compound, when administered, the compound will target and concentration at a spinal fusion in the patient (if present) and, where X is a diagnostic agent, a physician or other healthcare provider can easily image and/or identify the imaging agent of the compound.

In some embodiments, X is a therapeutic agent for treating a spinal fusion in the patient. In some embodiments, X is any suitable bone anabolic agent. In some embodiments, the bone anabolic agent is neutral, anionic, cationic, or hydrophobic. In some embodiments, the bone anabolic agent is an oligopeptide (e.g., comprising less than or equal to about 10 (or less than 10) amino acid residues, such as 10, 9, 8, 7, 6, 5 or 4 amino acid residues). In some embodiments, the bone anabolic agent comprises more than or equal to about 10 (or more than 10) amino acid residues, such as 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acid residues.

Examples of anabolic agents include, but are not limited to, abaloparatide, preptin (e.g., a 34-residue peptide hormone that is secreted by the β-cells of the pancreatic islets; corresponds to Asp69-Leu102 of the E-peptide of proinsulin-like growth factor II (pro-IGF-II)), integrin 5 beta 1 (ITGA), dasatinib, PTH, PTHrP, or a derivative of any of the foregoing (e.g., one or more amino acid mutations, such as insertions, deletions, and substitutions with a naturally occurring amino acid or a non-naturally occurring amino acid) having bone anabolic activity, or a fragment of any of the foregoing having bone anabolic activity.

In some embodiments, a bone anabolic agent followed by the designation D #, such as, e.g., D20, indicates that the bone anabolic agent is attached (or joined or connected, such as at an N-terminus or a C-terminus) to an osteotropic ligand (e.g., such as an orthotropic ligand having aspartic acid residues). In some embodiments, a bone anabolic agent followed by the designation E #, e.g., E20, indicates that the bone anabolic agent is attached (or joined or connected, such as at an N-terminus or a C-terminus) to an osteotropic ligand (e.g., such as an orthotropic ligand having 20 glutamic acid residues).

In some embodiments of Formula (I), Y is a non-releasable linker. In some embodiments, Y is a non-releasable linker containing at least one carbon-carbon bond, at least one amide bond, or at least one carbon-carbon bond and at least one amide bond. In some embodiments, Y is a releasable linker. In some embodiments, Y is a releasable linker containing at least one disulfide bond (S—S), at least one ester (e.g., —O(C═O)—), at least one protease-specific amide bond, or a combination of one or more of the foregoing.

In some embodiments, the targeting molecule (i.e., osteotropic ligand (Z)) does not cleave from the drug/anabolic agent (X) for the compound to be therapeutically effective in vivo. This can be advantageous as it can allow for the use of osteotropic ligands and compositions comprising anabolic agents because only a negligible amount (if any) of the anabolic agent is released (e.g., systemically) prior to the targeted delivery of the compound to the spinal fusion site. In some embodiments, tuning the releasing properties of active components is a difficult aspect of the preparation of effective pharmaceutical compositions. In some embodiments, the compounds comprising the non-releasable linkers provided herein avoid the difficulties of the preparation of effective pharmaceutical compositions (e.g., by removing the necessity of timing the release). In some embodiments, the anabolic agent of the compound provided herein is active when bound (e.g., conjugated to the osteotropic ligand). Accordingly, in some embodiments, the compounds comprising targeting molecules (e.g., Z) conjugated with a non-releasable linker (e.g., Y) can reduce systemic exposure and/or systemic adverse effects of the anabolic agents (X) linked therewith.

In some embodiments, a conjugate comprising a non-releasable linker reduces or eliminates toxicity of a component released from the conjugate in its free form (e.g., a free form of a compound and/or ligand provided herein).

Both releasable and non-releasable linkers can be engineered to optimize biodistribution, bioavailability, and PK/PD (e.g., of the compound) and/or to increase uptake (e.g., of the compound) into the targeted tissue pursuant to methodologies commonly known in the art or hereinafter developed such as through PEGylation and the like. In some embodiments, the linker is configured to avoid significant release of a pharmaceutically active amount of the anabolic agent in circulation prior to capture by a cell (e.g., a bone cell at the spinal fusion site).

In some embodiments, linkers can comprise one or more spacers (e.g., to facilitate a particular release time, facilitate an increase in uptake into a targeted tissue, and/or optimize biodistribution, bioavailability, and/or PK/PD of a compound). A spacer can comprise one or more of alkyl chains, polyethylene glycols (PEGs), peptides, sugars, peptidoglycans, clickable linkers (e.g., triazoles), rigid linkers such as poly-prolines and poly-piperidines, and the like.

In some embodiments, the one or more linkers of the compounds provided herein can comprise PEG, a PEG derivative, or any other linker known in the art or hereinafter developed that can achieve the purpose set forth herein. In some embodiments, the linker is repeated n times, where n is a positive integer.

In some embodiments, X is abaloparatide, ITGA, dasatinib, PTH, PTHrP, or a derivative or fragment of any of the foregoing having bone anabolic activity; Y is a non-releasable oligopeptide linker; and Z is DE20. In some embodiments, X is abaloparatide or a derivative or fragment thereof having bone anabolic activity; Y is a releasable oligopeptide linker comprising at least one protease-specific amide bond; and Z is DE20.

In some embodiments, the compound is any compound, or a pharmaceutically acceptable salt thereof, provided herein (e.g., a compound having a structure of Formula (I), SEQ ID NO: 1, or SEQ ID NO: 2). The letter “H” at the beginning of a peptide sequence indicates the N-terminus. For example, SEQ ID NO: 1, which is: HAVSEHQLLHDKGKSIQDLRRRELLEKLLxKLHTAEIRATSEVSPNSeeeeeeeeee, wherein x=α-aminoisobutyric acid (Aib), “e” signifies D-glutamic acid, and H indicates the adjacent Alanine residue is an N-terminal Alanine residue, such that SEQ ID NO: 1 can be understood as H-AVSEHQLLHDKGKSIQDLRRRELLEKLLxKLHTAEIRATSEVSPNSeeeeeeeeee (or alternatively and, as presented in the computer readable form of the Sequence Listing submitted concurrently herewith in accordance with WIPO Standard ST.25 (1998), Appendix 2, Tables 1 and 3, SEQ ID NO: 1 is AVSEHQLLHDKGKSIQDLRRRELLEKLLxKLHTAEIRATSEVSPNSeeeeeeeeee, wherein Alanine is the N-terminal residue, and “x” and “e” are as defined herein).

Similarly, SEQ ID NO: 2, which is HAVSEHQLLHDKGKSIQDLRRRELLEKLLxKLHTAEIRATSEVSPNSeeeeeeeeeeeeeeeeeee e, wherein x=Aib and “e” signifies D-glutamic acid, the H at the beginning of the sequence indicates the N-terminal end of the sequence, such that SEQ ID NO: 2 can be understood as H-AVSEHQLLHDKGKSIQDLRRRELLEKLLxKLHTAEIRATSEVSPNSeeeeeeeeeeeeeeeeeeee (or alternatively and, as presented in the computer readable form of the Sequence Listings submitted concurrently herewith in accordance with WIPO Standard ST.25 (1998), Appendix 2, Tables 1 and 3, SEQ ID NO: 2 is AVSEHQLLHDKGKSIQDLRRRELLEKLLxKLHTAEIRATSEVSPNSeeeeeeeeeeeeeeeeeeee, wherein the N-terminal amino acid is Alanine, and “x” and “e” are as defined herein). In some embodiments, “E” corresponds to L-glutamic acid and “e” corresponds to D-glutamic acid.

In some embodiments, the compound has at least 75% sequence identity or more, at least 85% sequence identity or more, at least 90% sequence identity or more, or at least 95% sequence identity or more to SEQ ID NO: 1. In some embodiments, the compound has at least 75% sequence identity or more, at least 85% sequence identity or more, at least 90% sequence identity or more, or at least 95% sequence identity or more to SEQ ID NO: 2.

Compounds can be synthesized in accordance with methods known in the art and exemplified herein, such as solid phase peptide synthesis.

Pharmaceutical Compositions

The compounds described herein can be administered alone or formulated as a pharmaceutical composition comprising the compound or compounds and one or more pharmaceutically acceptable excipients. The term “composition” generally refers to any product comprising more than one ingredient, including the compounds described herein. It is to be understood that the compositions described herein can be prepared from isolated compounds or from salts, solutions, hydrates, solvates, and other forms of the compounds. Certain functional groups, such as the hydroxy, amino, and like groups, can form complexes with water and/or various solvents, in the various physical forms of the compounds. It is also to be understood that the compositions can be prepared from various amorphous, non-amorphous, partially crystalline, crystalline, and/or other morphological forms of the compounds, and the compositions can be prepared from various hydrates and/or solvates of the compounds. Accordingly, such pharmaceutical compositions that recite compounds include each of, or any combination of, or individual forms of, the various morphological forms and/or solvate or hydrate forms of the compounds.

One embodiment provides a pharmaceutical composition comprising a compound of Formula (I) or any compound covered by such formula, or a pharmaceutically acceptable salt thereof, and at least one pharmaceutically acceptable carrier and/or excipient.

One embodiment provides a pharmaceutical composition comprising an effective amount of a therapeutically (or prophylactically) effective compound of Formula (I) or any compound covered by such formula, or a pharmaceutically acceptable salt thereof, and at least one pharmaceutically acceptable carrier and/or excipient.

In some embodiments, provided herein is a pharmaceutical composition comprising a therapeutically effective amount of any compound provided herein that can be administered (e.g., subcutaneously) to a patient in need thereof. In various embodiments, the composition is an injectable composition, such as a composition that is suitable for subcutaneous injection.

Compounds and/or compositions described herein may be administered in unit dosage forms and/or compositions containing one or more pharmaceutically acceptable carriers, adjuvants, diluents, excipients, and/or vehicles, and combinations thereof.

The term “administering” generally refers to any and all means of introducing compounds described herein to the host subject including, but not limited to, by oral, intravenous, intramuscular, subcutaneous, transdermal, inhalation, buccal, ocular, sublingual, vaginal, rectal, and like routes of administration.

Administration of the compounds as salts can be appropriate. Examples of acceptable salts include, without limitation, alkali metal (for example, sodium, potassium or lithium) or alkaline earth metals (for example, calcium) salts; however, any salt that is generally non-toxic and effective when administered to the subject being treated is acceptable. In at least one embodiment, the salt can be ammonium acetate salt. Similarly, “pharmaceutically acceptable salt” refers to those salts with counter ions which may be used in pharmaceuticals. Such salts may include, without limitation: (1) acid addition salts, which can be obtained by reaction of the free base of the parent compound with inorganic acids, such as hydrochloric acid, hydrobromic acid, nitric acid, phosphoric acid, sulfuric acid, perchloric acid, and the like, or with organic acids, such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid, malonic acid, and the like; or (2) salts formed when an acidic proton present in the parent compound either is replaced by a metal ion, e.g., an alkali metal ion, an alkaline earth ion, or an aluminum ion, or coordinates with an organic base, such as ethanolamine, diethanolamine, triethanolamine, trimethamine, N-methylglucamine, and the like. Pharmaceutically acceptable salts are well-known to those skilled in the art, and any such pharmaceutically acceptable salts are contemplated.

Acceptable salts can be obtained using standard procedures known in the art, including (without limitation) reacting a sufficiently acidic compound with a suitable base, affording a physiologically acceptable anion. Suitable acid addition salts are formed from acids that form non-toxic salts. Illustrative, albeit nonlimiting, examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts. Suitable base salts of the compounds described herein are formed from bases that form non-toxic salts. Illustrative, albeit nonlimiting, examples include the arginine, benzathine, calcium, choline, diethylamine, diolamine, glycine, lysine, magnesium, meglumine, olamine, potassium, sodium, tromethamine and zinc salts. Hemi-salts of acids and bases, such as hemisulphate and hemicalcium salts, also can be formed.

The compounds can be formulated as pharmaceutical compositions and administered to a mammalian host, such as a human patient, in a variety of forms adapted to the chosen route of administration. For example, the pharmaceutical composition can be formulated for and administered via intraosseous, intravenous, intraarterial, intraperitoneal, intracranial, intramuscular, topical, inhalation and/or subcutaneous routes. In at least one embodiment, a compound and/or composition can be administered directly (via injection, placement or otherwise) to a spinal fusion site. In at least one embodiment, the compounds can be systemically administered in combination with a pharmaceutically acceptable vehicle, such as an inert diluent or an assimilable edible carrier. For oral therapeutic administration, the active compound can be combined with one or more excipients and used in the form of ingestible tablets, buccal tablets, troches, capsules, elixirs, suspensions, syrups, wafers, and the like. The percentage of the compositions and preparations can vary and may be between about 1 to about 99% weight of the active ingredient(s) and a binder, excipients, a disintegrating agent, a lubricant, and/or a sweetening agent (as are known in the art). The amount of active compound in such therapeutically useful compositions is such that an effective dosage level will be obtained.

The preparation of parenteral compounds/compositions under sterile conditions, for example, by lyophilization, can readily be accomplished using standard pharmaceutical techniques well-known to those skilled in the art. In at least one embodiment, the solubility of a compound used in the preparation of a parenteral composition can be increased by the use of appropriate formulation techniques, such as the incorporation of solubility-enhancing agents.

As previously noted, the compounds/compositions can also be administered via infusion or injection (e.g., using needle (including microneedle) injectors and/or needle-free injectors). Solutions of the active composition can be aqueous, optionally mixed with a nontoxic surfactant and/or contain carriers or excipients, such as salts, carbohydrates and buffering agents (preferably at a pH of from 3 to 9), but, for some applications, they may be more suitably formulated as a sterile non-aqueous solution or as a dried form to be used in conjunction with a suitable vehicle, such as sterile, pyrogen-free water or phosphate-buffered saline. For example, dispersions can be prepared in glycerol, liquid PEGs, triacetin, and mixtures thereof and in oils. Under ordinary conditions of storage and use, these preparations can further contain a preservative to prevent the growth of microorganisms.

The pharmaceutical dosage forms suitable for injection or infusion can include sterile aqueous solutions or dispersions or sterile powders comprising the active ingredients that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example and without limitation, water, ethanol, a polyol (e.g., glycerol, propylene glycol, liquid PEG(s), and the like), vegetable oils, nontoxic glyceryl esters, and/or suitable mixtures thereof. In at least one embodiment, the proper fluidity can be maintained by the formation of liposomes, by the maintenance of the required particle size in the case of dispersions or by the use of surfactants. The action of microorganisms can be prevented by the addition of various antibacterial and antifungal agents, such as parabens, chlorobutanol, phenol, sorbic acid, thimerosal, and the like. In certain cases, it can be desirable to include one or more isotonic agents, such as sugars, buffers, or sodium chloride. Prolonged absorption of the injectable compositions can be brought about by the incorporation of agents formulated to delay absorption, for example, aluminum monostearate and gelatin.

Sterile injectable or infusible solutions can be prepared by incorporating the active compound and/or composition in the required amount of the appropriate solvent with one or more of the other ingredients set forth above, as required, followed by filter sterilization. In the case of sterile powders for the preparation of sterile injectable solutions, the preferred methods of preparations are vacuum drying and freeze-drying, which yield a powder of the active ingredient plus any additional desired ingredient present in the previously sterile-filtered solutions.

For topical administration, it can be desirable to administer the compounds to the bone as compositions or formulations in combination with an acceptable carrier, which may be a solid, a liquid, or a gel matrix. For example, in certain embodiments, useful liquid carriers can comprise water, alcohols or glycols or water-alcohol/glycol blends, in which the compounds can be dissolved or dispersed at effective levels, optionally with the aid of non-toxic surfactants. In some embodiments, the compounds and/or compositions can be administered topically on a collagen sponge, a mineralized collagen, or a bone graft.

Additionally or alternatively, adjuvants, such as antimicrobial agents, can be added to optimize the properties for a given use. The resultant liquid compositions can be applied from absorbent pads, used to impregnate bandages and/or other dressings, sprayed onto the targeted area using pump-type or aerosol sprayers, or simply applied directly to a desired area of the subject (e.g., a spinal fusion surgical site).

Thickeners, such as synthetic polymers, fatty acids, fatty acid salts and esters, fatty alcohols, modified celluloses or modified mineral materials can also be employed with liquid carriers to form spreadable pastes, gels, ointments, soaps, and the like for application directly to the skin of the subject.

As used herein, the term “therapeutically effective dose” means (unless specifically stated otherwise) a quantity of a compound which, when administered either one time or over the course of a treatment cycle affects the health, well-being or mortality of a subject (e.g., and without limitation, supports or promotes the healing of the spinal fusion site and/or bone growth or fusion).

In some embodiments, the therapeutically effective amount of any compound or pharmaceutical composition provided herein is determined in accordance with methods known in the art (e.g., animal models, human data, and human data for compounds that exhibit similar pharmacological activities). Useful dosages of the compounds can be determined by comparing their in vitro activity and the in vivo activity in animal models. Methods of the extrapolation of effective dosages in mice and other animals to human subjects are known in the art. Indeed, the dosage of the compound can vary significantly depending on the condition of the host subject, the bone fracture being treated, the route of administration of the compound and tissue distribution, and the possibility of co-usage of other therapeutic treatments (for example, in conjunction with the administration of other injectable compositions for promoting bone growth such as growth factors, stem cells, natural grafts, biologic- and synthetic-based tissue-engineered scaffolds and the like, hardware implantation, and/or ultrasound therapies and the like). In some embodiments, the therapeutically effective amount of any compound or pharmaceutical composition provided herein is determined by taking into consideration, for example, the potency of X of Formula (I) (e.g., the type of therapeutic agent employed), body weight, mode of administration (e.g., subcutaneously), disease or condition being treated, disease or condition its severity, the like, or any combination thereof. The amount of the composition required for use in treatment (e.g., the therapeutically effective amount or dose) will vary not only with the particular application, but also with the salt selected (if applicable) and the characteristics of the subject (such as, for example, age, condition, sex, the subject's body surface area and/or mass, tolerance to drugs) and will ultimately be at the discretion of the attendant physician, clinician, or otherwise.

In some embodiments, the therapeutically effective amount of any compound or pharmaceutical composition provided herein is from about 0.01 mg/kg/day up to about 1,000 mg/kg/day. For example, therapeutically effective amounts or doses can range from about 0.05 mg/kg of patient body weight to about 30.0 mg/kg of patient body weight, or from about 0.01 mg/kg of patient body weight to about 5.0 mg/kg of patient body weight, including but not limited to 0.01 mg/kg, 0.02 mg/kg, 0.03 mg/kg, 0.04 mg/kg, 0.05 mg/kg, 0.1 mg/kg, 0.2 mg/kg, 0.3 mg/kg, 0.4 mg/kg, 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, and 5.0 mg/kg, all of which are kg of patient body weight. Intravenous doses can be several orders of magnitude lower. In some embodiments, the compound the therapeutically effective amount of any compound or pharmaceutical composition provided herein is administered (e.g., subcutaneously) daily, weekly, bi-weekly, monthly, or bi-monthly.

In some embodiments, the therapeutically effective amount (e.g., administered to the individual) of any compound or pharmaceutical composition provided herein (e.g., SEQ ID NO: 1 or SEQ ID NO: 2) is from about 0.01 mg/kg/day up to about 1,000 mg/kg/day. In some embodiments, the therapeutically effective amount (e.g., administered to the individual) of any compound or pharmaceutical composition provided herein (e.g., SEQ ID NO: 1 or SEQ ID NO: 2) is about 1 μg/dose to about 10 mg/dose. In some embodiments, the therapeutically effective amount (e.g., administered to the individual) of any compound or pharmaceutical composition provided herein (e.g., SEQ ID NO: 1 or SEQ ID NO: 2) is about 50 μg/dose to about 5 mg/dose. In some embodiments, the therapeutically effective amount (e.g., administered to the individual) of any compound or pharmaceutical composition provided herein (e.g., SEQ ID NO: 1 or SEQ ID NO: 2) is about 0.01 nmol/kg/dose to about 10 ng/kg/dose. In some embodiments, the therapeutically effective amount (e.g., administered to the individual) of any compound or pharmaceutical composition provided herein (e.g., SEQ ID NO: 1 or SEQ ID NO: 2) is about 0.1 nmol/kg/dose to about 5 ng/kg/dose.

The total therapeutically effective amount of the compound can be administered in single or divided doses and can, at the practitioner's discretion, fall outside of the typical range given herein. In some embodiments, a therapeutically effective amount of any compound or pharmaceutical composition provided herein is administered once or twice weekly. In some embodiments, a therapeutically effective amount of any compound or pharmaceutical composition provided herein is administered once weekly. In some embodiments, a therapeutically effective amount of any compound or pharmaceutical composition provided herein is administered twice weekly.

The effective amount of an X-Y-Z conjugate, such as a pharmaceutical composition comprising an effective amount of the conjugate, can be administered by any suitable route (e.g., subcutaneously). An example of a suitable route is by injection, such as subcutaneous injection.

Kits

In some embodiments, provided herein is a kit for treating a spinal fusion in a patient and/or targeting a therapeutic agent or diagnostic agent to a spinal fusion in the patient. In some embodiments, the kit comprises:
(a) either:
   (i) a compound of formula X-Y-Z, wherein:
   X is a therapeutic agent for treating a spinal fusion in the patient (e.g., a bone anabolic agent) or a diagnostic agent for identifying a spinal fusion in the patient,
   Y is absent or, when present, is a linker, which can be either releasable or non-releasable, and
   Z is an osteotropic ligand (e.g., an AOP comprising at least 4 amino acid residues to 20 amino acid residues), or
   (ii) a pharmaceutical composition comprising the compound or pharmaceutically acceptable salt thereof of subpart (i) and a pharmaceutically acceptable carrier; and

(b) a collagen sponge, a mineralized collagen, or a bone graft. In some embodiments, administration of a therapeutically effective amount of the compound having a structure of Formula (I) or a pharmaceutically acceptable salt thereof to the patient treats the spinal fusion.

The compound of the kit can be any compound or pharmaceutical composition described herein (e.g., a compound of Formula (I), SEQ ID NO: 1, or SEQ ID NO: 2). In some embodiments where X is a therapeutic agent, it can be a bone anabolic agent that is PTH, a PTHrP, a derivative of either of the foregoing having bone anabolic activity, or a fragment of any of the foregoing having bone anabolic activity. In some embodiments where X is a therapeutic agent (e.g., a bone anabolic agent), X can be abaloparatide, a derivative thereof having bone anabolic activity, or a fragment thereof having bone anabolic activity, or any other suitable bone anabolic agent. In some embodiments, X is a therapeutic agent selected from the group consisting of abaloparatide, ITGA, dasatinib, PTH, PTHrP, and a derivative or fragment of any of the foregoing having bone anabolic activity.

In some embodiments, X of the compound or pharmaceutical composition is a diagnostic agent. Such a diagnostic agent can be any suitable imaging agent such that administration of the compound having a structure of Formula (I) or a pharmaceutically acceptable salt thereof (or, a pharmaceutical composition comprising such a compound or pharmaceutically acceptable salt thereof) identifies the spinal fusion if present. In some embodiments, the diagnostic agent is a fluorescent dye. In some embodiments, the diagnostic agent is a near-infrared fluorescent molecule.

Methods of Treatment

Provided in some embodiments herein are methods of treating a spinal fusion (e.g., in an individual in need thereof). Provided in some embodiments herein are methods of treating a spinal fusion in a patient (e.g., in need thereof) using the compounds and/or compositions provided. The compounds, compositions and methods can leverage strategies to (e.g., selectively) target the spinal fusion site to prevent off-target effects of the therapeutic (e.g., anabolic) agents present within the compounds or compositions.

In some embodiments, the method comprises administering (e.g., subcutaneously) (e.g., to a patient in need thereof) a therapeutically effective amount of any of the compounds or pharmaceutical compositions described herein including, for example:

(i) a compound provided herein, such as, for example, a compound having a structure of Formula (I):

X-Y-Z,
or a pharmaceutically acceptable salt thereof, wherein:
   X is a bone anabolic agent,
   Y is absent or, when present, is a linker, which can be either releasable or non-releasable, and
   Z is an osteotropic ligand, which can be, for example, an AOP (e.g., comprising at least 4 amino acid residues to 20 amino acid residues, or
(ii) a pharmaceutical composition comprising the compound or pharmaceutically acceptable salt thereof of subpart (i) and a pharmaceutically acceptable carrier.

Methods for localizing a therapeutic agent or a diagnostic agent to a spinal fusion in a patient are also described. In some embodiments, a method for localizing a therapeutic agent or a diagnostic agent to a spinal fusion site in a patient comprises administering to the patient a therapeutically effective amount of any compound or pharmaceutically acceptable salt thereof described herein.

As shown in FIG. 2, provided herein is a biodistribution of targeted near-infrared (IR) fluorescent molecule 50456 (see, e.g., Scheme 1 in Example 9 below) 24 hours post-injection in a bilateral posterolateral lumbar spinal fusion with a collagen scaffold two weeks post-surgery. The animals were imaged for 1 second with excitation at 745 nM and emission at 810 nM on an AMI spectral imaging instrument at 5% excitation power (see Examples 4 and 7 below).

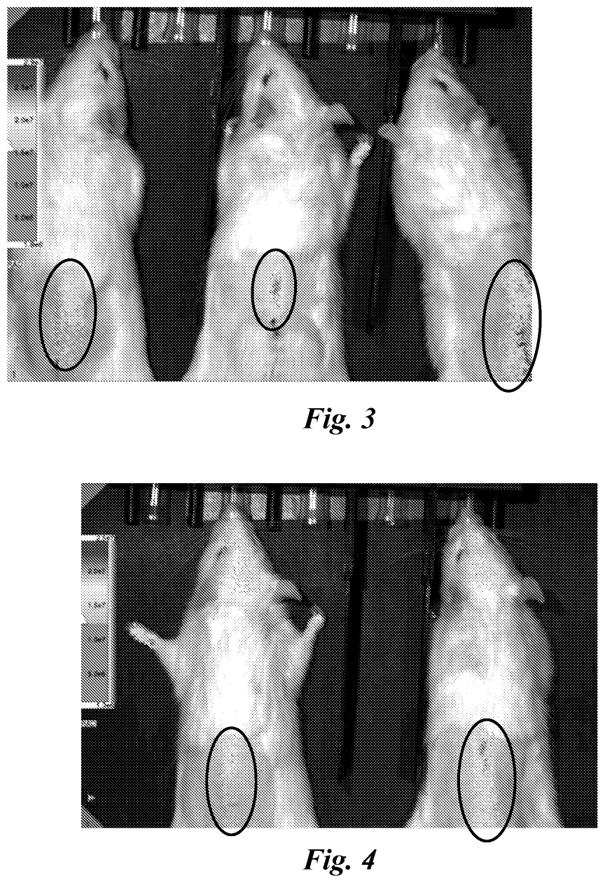

FIG. 3 shows the biodistribution of targeted near-IR fluorescent molecule S0456 24 hours post-injection in a bilateral posterolateral lumbar spinal fusion with a mineralized collagen scaffold two weeks post-surgery. The animals were imaged for 1 second with excitation at 745 nM and emission at 810 nM on an AMI spectral imaging instrument at 5% excitation power (see Examples 4 and 7 below).

FIG. 4 shows the biodistribution of targeted near-IR fluorescent molecule S0456 24 hours post-injection in a bilateral posterolateral lumbar spinal fusion with bone graft scaffold two weeks post surgery. In some embodiments, the animals are imaged for 1 second with excitation at 745 nM and emission at 810 nM on an AMI spectral imaging instrument at 5% excitation power (see Examples 4 and 7 below).

In some embodiments, as shown in FIG. 4, localization to paws and head were on the exterior, which was due to the compound appearing in the urine (e.g., which the animals walked in and then rubbed on their heads during grooming).

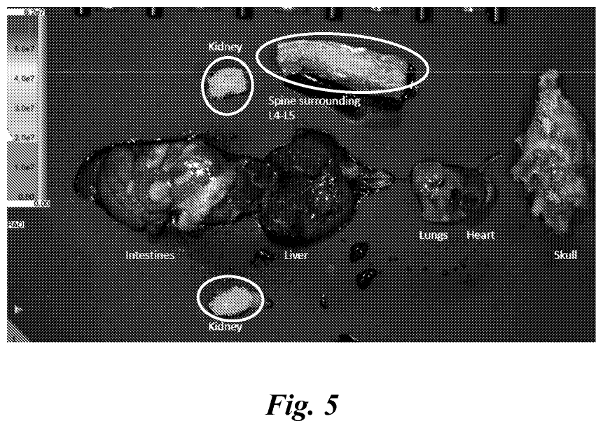

FIG. 5 shows the biodistribution of targeted near-IR fluorescent molecule S0456 24 hours post-injection in bilateral posterolateral lumbar spinal fusion with collagen scaffold eight weeks post-surgery. There, the animals were subjected to a full necropsy to check for accumulation in other (non-targeted) tissues. The tissues were imaged for 1 second with excitation at 745 nM and emission at 810 nM on an AMI spectral imaging instrument at 5% excitation power.

In some instances, localization of a compound provided herein (e.g., the targeted compound) to the spinal fusion site was observed with all scaffold types. In some embodiments, full-body dissection (see FIG. 5) revealed only localization in the kidneys and the spine. As the kidneys are the excretion pathway for certain embodiments of the compounds provided herein, these results suggest that acidic oligopeptides can be used to localize therapeutic to spinal fusions. In some embodiments, the localization on the paws and the head were due to the compound being excreted through the urine (e.g., which the rats stepped in and then rubbed on their heads during grooming).

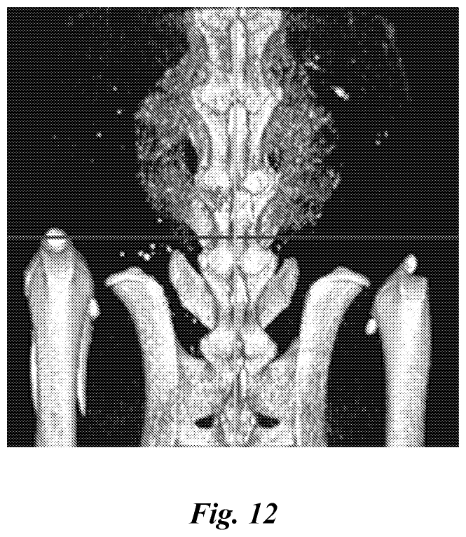

FIG. 12 shows a three-dimensional reconstructions of micro-CT scans of a posterolateral spinal fusion of 14-15 in a rat treated with a mineralized collagen scaffold and saline one week post-implantation. In some embodiments, the mineralization was inherent to the scaffold (e.g., which was visible early in the spinal fusion process).

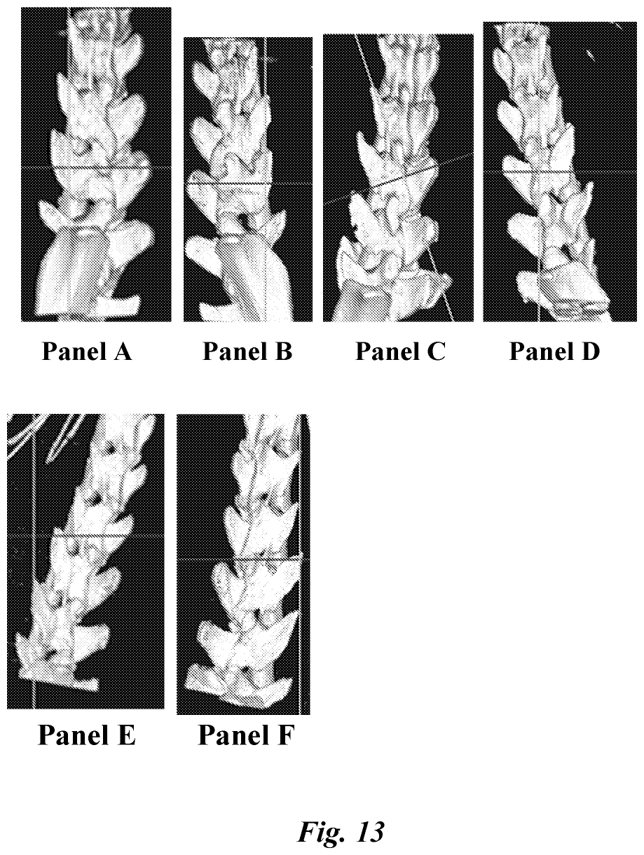

In some embodiments, the scaffolds resorbed too quickly, and after 8 weeks none of the treatments had fused. For example, FIG. 13 shows three-dimensional reconstructions of micro-CT scans of posterolateral spinal fusions of L4-L5 in rats treated with a mineralized collagen scaffold and different treatments of the compounds hereof (e.g., saline, BMP2 and abaloparatide DE20) after eight weeks of treatment. In some embodiments, the scaffolds resorbed too fast to allow for bridging. The three-dimensional reconstructions of micro-CT scans shown in FIG. 13, panels A and B are for spinal fusion treated with mineralized collagen and BMP2 after eight weeks. The three-dimensional reconstructions of micro-CT scans shown in FIG. 13, panels C and D are for spinal fusion treated with mineralized collagen and SEQ ID NO: 2 after eight weeks. The three-dimensional reconstructions of micro-CT scans shown in FIG. 13, panels E and F are for spinal fusion treated with mineralized collagen and saline after eight weeks.

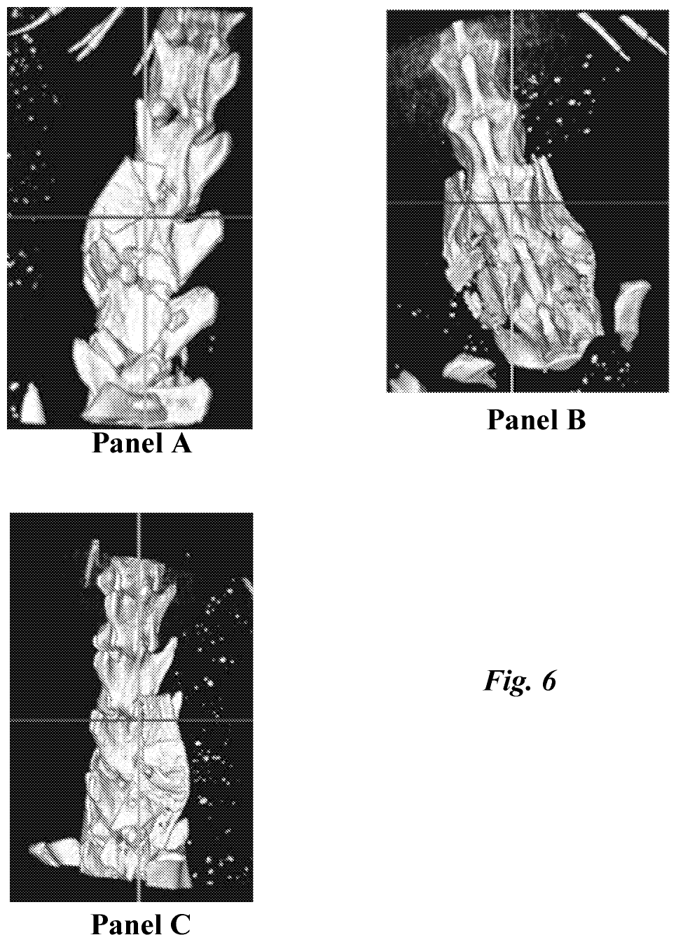

In some embodiments, transverse processes healed from the decortication. In some embodiments, no bone formed between the L4 and L5 vertebrae. However, the most promising and consistent were the collagen scaffolds of which only the abaloparatide (e.g., a compound of SEQ ID NO.: 2)-treated spines fused (e.g., FIG. 6).

FIG. 6, panels A-C show three-dimensional reconstructions of micro-CT scans of a posterolateral spinal fusion of L4-L5 in a rat treated with a plain collagen matrix and a compound of SEQ ID NO: 2 for eight weeks. In some embodiments, SEQ ID NO: 2 was dosed (e.g., in rodents) at 33 nmol/kg twice a week. Fusion was confirmed by manual palpitation.

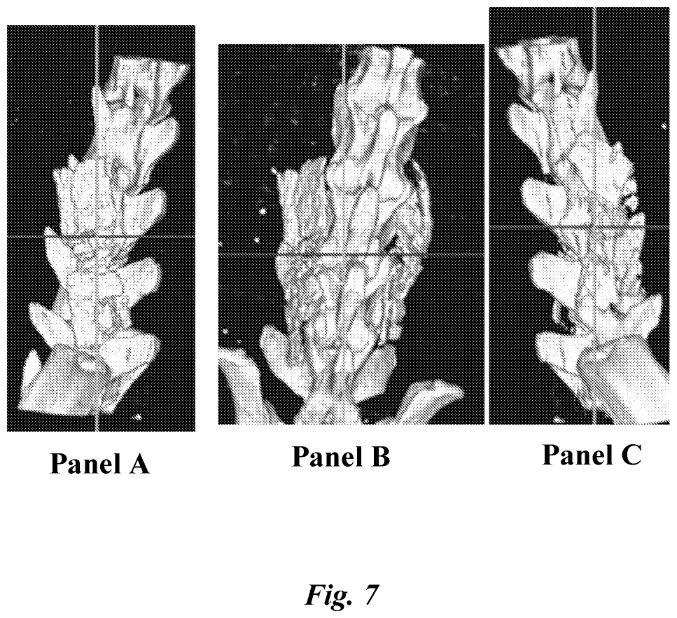

FIG. 7, panels A-C show the three-dimensional reconstructions of micro-CT scans of a posterolateral spinal fusion of L4-L5 in a rat treated with a plain collagen matrix soaked with rhBMP-2 (10 μg) for eight weeks. In some embodiments, the collagen matrix was applied during surgery. FIG. 7, panels A-C show the BMP-treated spines (e.g., the positive control) did ossify, but not in as controlled of a manner as those treated with a compound of SEQ ID NO: 2 (e.g., possibly due to ectopic leakage or poor decortication). In some embodiments, the BMP-treated spines did not fuse at all (manual palpitation confirmed no fusion).

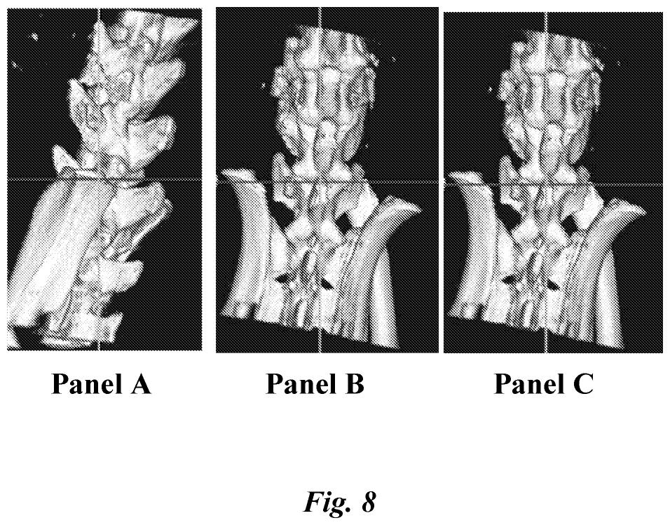

In some embodiments, the collagen scaffold and the surgery alone are not sufficient for bone formation. In some embodiments, an anabolic composition is sufficient to mineralize the sponge. In some embodiments, the bone grafts fused completely (e.g., which could be an artifact of the difficulty in packing the sponge with enough bone granules consistently) (e.g., FIG. 8).

FIG. 8, panels A-C shows three-dimensional reconstructions of micro-CT scans of a posterolateral spinal fusion of L4-L5 in a rat treated with a plain collagen matrix and saline for eight weeks. In some embodiments, phosphate-buffered saline was dosed at a 1× concentration twice a week in the same volume as SEQ ID NO: 2. In some embodiments, manual palpitation confirmed no fusion in the phosphate-buffered saline-treated rats.

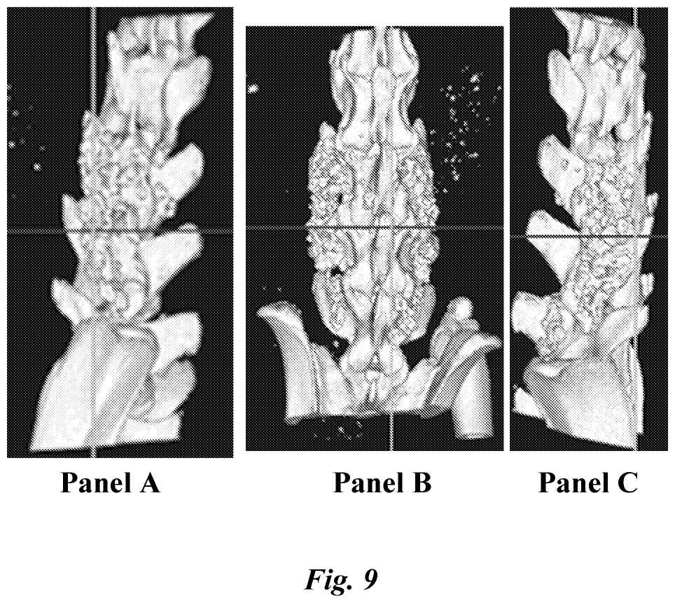

In some embodiments, the BMP caused some of the granules to fuse (e.g., FIG. 9, panels A-C).

FIG. 9, panels A-C show three-dimensional reconstructions of micro-CT scans of a posterolateral spinal fusion of L4-L5 in a rat treated with a bone graft matrix and rhBMP-2 (10 μg) for eight weeks. rhBMP-2 was soaked into the collagen sponge bone graft matrix applied during surgery. In some embodiments, manual palpitation confirmed no fusion.

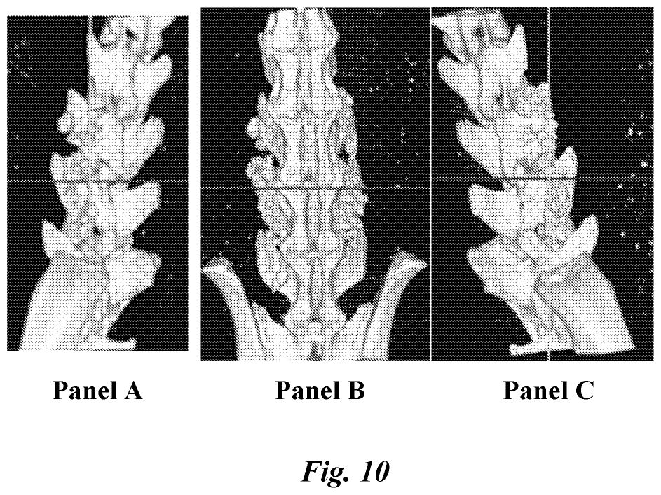

In some embodiments, the granules resorb in the abaloparatide-treated rats (FIG. 10, panels A-C).

In some embodiments, provided herein are three-dimensional reconstructions of micro-CT scans of a posterolateral spinal fusion of L4-L5 in a rat treated with a bone graft matrix and SEQ ID NO: 2 for eight weeks. In some embodiments, SEQ ID NO: 2 is soaked into the collagen spone bone graft matrix applied during surgery. In some embodiments, SEQ ID NO: 2 was dosed at 33 nmol/kg twice a week (in rodents). In some embodiments, manual palpitation confirmed no fusion.

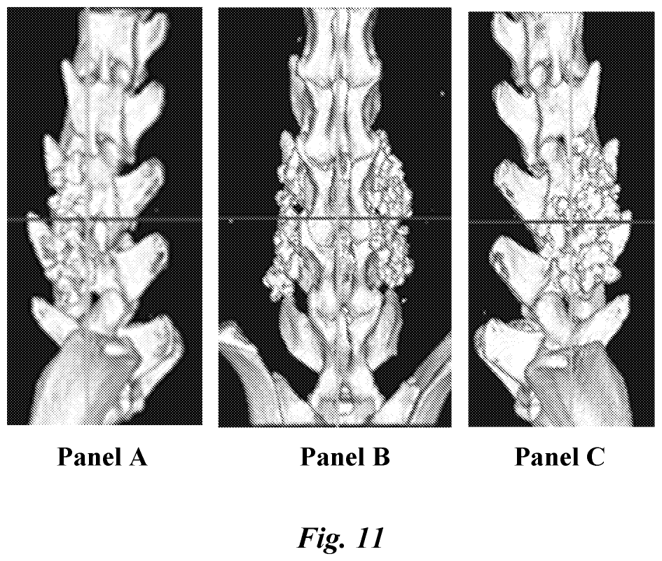

In some embodiments, the saline-treated rodents with bone grafts appeared to have no change in the number or shape of the granules (e.g., suggesting no change) (e.g., FIG. 11).

FIG. 11, panel A-C show three-dimensional reconstructions of micro-CT scans of a posterolateral spinal fusion of L4-L5 in a rat treated with a bone graft matrix and saline for eight weeks. In some embodiments, saline is soaked into the collagen spone bone graft matrix applied during surgery. In some embodiments, phosphate-buffered saline is dosed at a 1× concentration twice a week (in rodents) in the same volume as SEQ ID NO: 2.

In some embodiments, collagen scaffolds are used. In some instances, the collagen scaffolds, for example, produced consistent results, are commercially available, and commonly used in the clinic. While the mineralized collagen achieved targeting localization the fastest in regard to how much time post-spinal-fusion before the ability to target compounds was detected, it did not perform as expected in some cases. In some instances, spinal fusion took about 5 days for mineralized collagen, but in some instances, spinal fusion took closer to 12 days for collagen scaffolds. In some instances, spinal fusion took closer to 10 days for bone grafts. In some embodiments, for targeting, a blood supply and hydroxyapatite are needed.

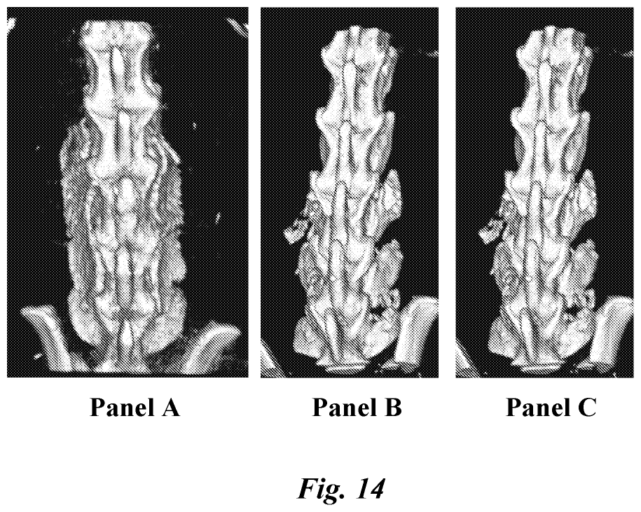

FIG. 14 shows representations of three-dimensional reconstructions of micro-CT scans of posterolateral spinal fusions of L4-L5 in rats treated with a mineralized collagen scaffold and different treatments of compounds of the present disclosure after five weeks of treatment (see Example 8 below). FIG. 14, panel A shows a three-dimensional reconstruction of micro-CT scans for a spinal fusion site taken from rats treated with mineralized collagen and SEQ ID NO: 2 after five weeks. FIG. 14, panel B shows a three-dimensional reconstruction of micro-CT scans for a spinal fusion site taken from rats treated with mineralized collagen and rhBMP-2 (10 μg) after five weeks. FIG. 14, panel C shows a three-dimensional reconstruction of micro-CT scans for a spinal fusion site taken from rats treated with mineralized collagen and saline after five weeks of treatment.

As shown in FIG. 14, administration of a compound of SEQ ID NO: 2 increased the mineralization of the scaffolds more than BMP2 or saline.

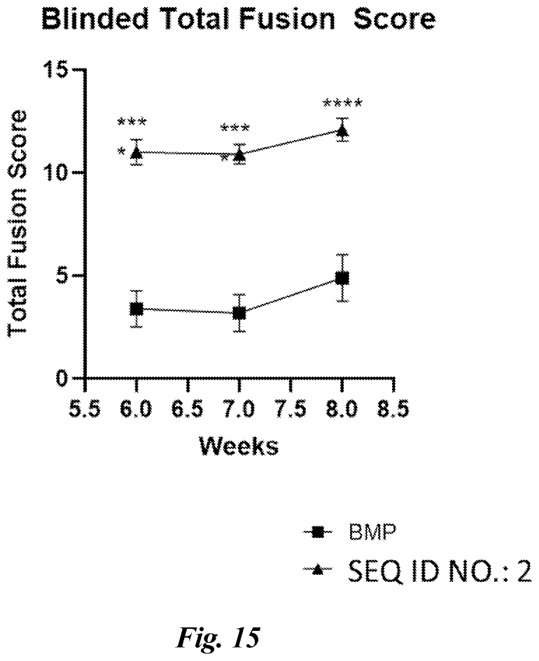

FIG. 15 shows a graph of weeks vs. total fusion score for BMP and SEQ ID NO: 2. Scores were assigned 0-5 on osteointegration, density, and bridging of both bilateral spinal fusions in microCT scans of the rats as determined by an individual who was blinded to the treatment and reviewed random samples. The total score is the sum of all five scores combined.

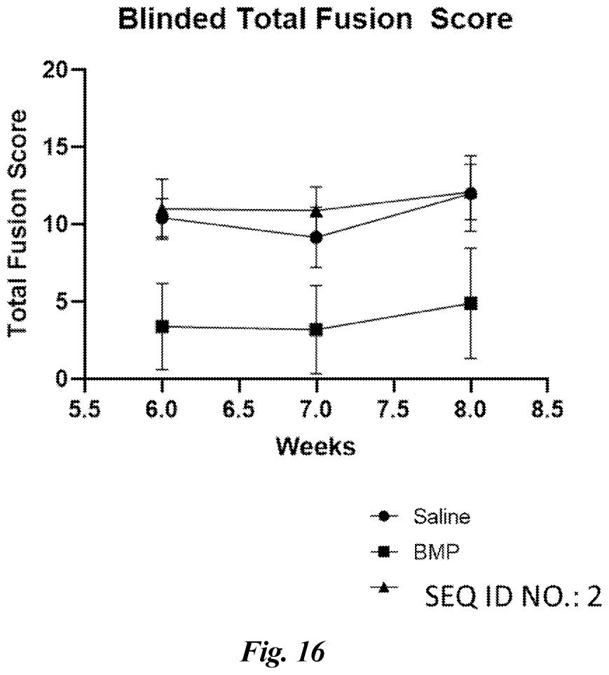

FIG. 16 provides a graph of weeks vs. total fusion score for saline, BMP and SEQ ID NO: 2. Scores are assigned 0-5 on osteointegration, density, bridging of both bilateral spinal fusions in microCT scans of the rats as determined by an individual who was blinded to the treatment and reviewed random samples. The total score is the sum of all five scores combined. In some instances, saline had an artificially high fusion due to the induction of a stronger osteogenic response by the removal of more bone during the decortication.

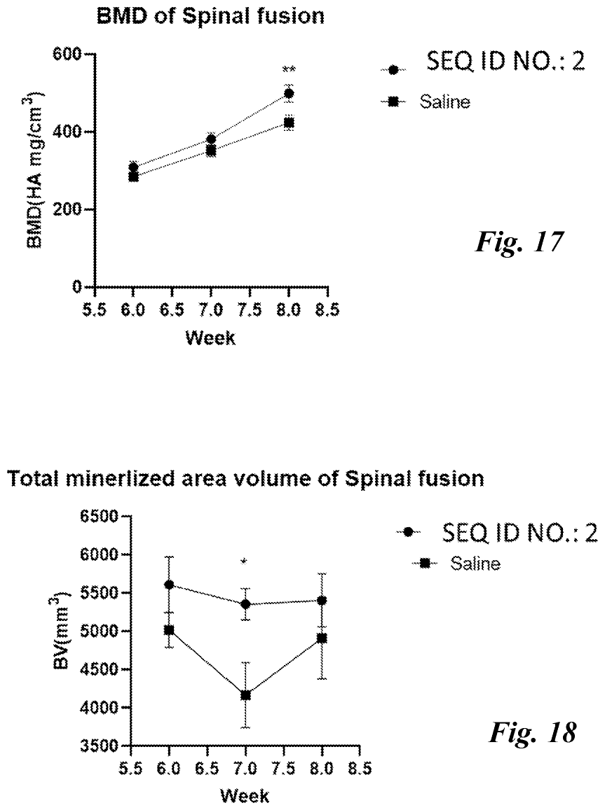

FIG. 17 shows a graph of weeks vs. BMD (bone mineral density; HA mg/cm 3) for SEQ ID NO: 2 and saline (e.g., which shows the BMD of the bone bridges formed during the fusion). In some instances, BMP did not generate enough mineralized tissue to be quantifiable. The density of the scan was assigned based on a standard curve generated by a phantom hydroxyapatite standard set.

FIG. 18 provides a graph of week vs. BV (bone volume; mm 3) (e.g., which shows the total area of mineralized tissue in the bone bridges formed by spinal fusions treated with SEQ ID NO: 2 or saline). This, in some instances, was quantified by ImageJ analysis of the microCT scans of the rats.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

While certain embodiments of the present disclosure have been shown and described herein, it will be apparent to those skilled in the art that such embodiments are provided by way of example only. It is not intended that the claimed invention be limited by the specific examples provided within the specification.

While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein, which depend upon a variety of conditions and variables. It should be understood that various alternatives to the embodiments of the invention described herein may be employed in practicing the invention. It is, therefore, contemplated that the invention shall also cover any such alternatives, modifications, variations or equivalents. It is intended that the following claims define the scope of the invention and that methods and structures within the scope of these claims and their equivalents be covered thereby.

### Certain Definitions

As used herein and in the appended claims, the singular forms “a,” “and,” and “the” include plural referents unless the context clearly dictates otherwise. Thus, for example, reference to “a compound” includes a plurality of such compounds. When ranges are used herein for physical properties, such as molecular weight, or chemical properties, such as chemical formulae, all combinations and sub-combinations of ranges and specific embodiments therein are intended to be included. The term “about,” when referring to a number or a numerical range, means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude an embodiment of any compound, composition, method, process, or the like that may “consist of” or “consist essentially of” the described features. The invention illustratively described herein may be suitably practiced in the absence of any element(s) or limitation(s), which is/are not specifically disclosed herein.

“Percent (%) sequence identity” with respect to a reference to a sequence is defined as the percentage of amino acid or nucleic acid residues, respectively, in a candidate sequence that are identical with the residues in the reference sequence, after aligning the sequences and introducing gaps, if necessary, to achieve the maximum percent sequence identity and not considering any conservative substitutions as part of the sequence identity. Alignment for purposes of determining percent sequence identity can be achieved in various ways that are within the skill of the art, for instance, using publicly available computer software. For example, determination of percent identity or similarity between sequences can be done, for example, by using the GAP program (Genetics Computer Group, software; now available via Accelrys on http://www.accelrys.com), and alignments can be done using, for example, the ClustalW algorithm (VNTI software, InforMax Inc., Gaithersburg, MD). Further, a sequence database can be searched using the nucleic acid or amino acid sequence of interest. Algorithms for database searching are typically based on the BLAST software (Altschul et al., 1990), but those skilled in the art can determine appropriate parameters for aligning sequences, including any algorithms needed to achieve maximal alignment over the full length of the sequences being compared. In some embodiments, the percent identity can be determined along the full-length of the nucleic acid or amino acid sequence.

As used herein, the terms “patient,” “subject,” and “individual” are used interchangeably. None of the terms require the supervision of medical personnel. For example, administering to an individual includes the individual administering the therapeutic agent to themselves, as well as a medical professional administering the therapeutic agent to the individual.

The term “radical” as used herein refers to a fragment of a molecule, wherein that fragment has an open valence which is an attachment point for bond formation. A monovalent radical has one open valence such that it can form one bond with another chemical group. In some embodiments, a radical of a molecule as used herein is created by removal of one hydrogen atom from that molecule to create a monovalent radical with one open valence at the location where the hydrogen atom was removed. Where appropriate, a radical can be divalent, trivalent, etc., wherein two, three or more hydrogen atoms have been removed to create a radical which can bond to two, three, or more chemical groups. Where appropriate, a radical open valence can be created by removal of other than a hydrogen atom (e.g., a halogen atom), or by removal of two or more atoms (e.g., a hydroxyl group), as long as the atoms removed are a small fraction (about 20% or less of the atom count) of the total atoms in the molecule forming the radical.

The terms “treat,” “treating,” or “treatment” include reducing, alleviating, abating, ameliorating, relieving, or lessening the symptoms associated with a bone fracture, diabetes, osteoporosis in either a chronic or acute therapeutic scenario.

The terms and expressions, which have been employed, are used as terms of description and not of limitation. In this regard, where certain terms are defined under “Certain Definitions” and are otherwise defined, described, or discussed elsewhere in the “Detailed Description,” all such definitions, descriptions, and discussions are intended to be attributed to such terms. There also is no intention in the use of such terms and expressions of excluding any equivalents of the features shown and described or portions thereof. Furthermore, while subheadings, e.g., “Certain Definitions,” are used in the “Detailed Description,” such use is solely for ease of reference and is not intended to limit any disclosure made in one section to that section only; rather, any disclosure made under one subheading is intended to constitute a disclosure under each and every other subheading.

### EXAMPLES

The following examples serve to illustrate the present disclosure. The examples are not intended to limit the scope of the claimed invention in any way.

### Example 1: Synthesis of Peptidic Payloads

All payloads (e.g., FIG. 1A) were synthesized in a solid-phase peptide synthesis vial under a stream of argon. Wang resin (0.6 mmol/g) was loaded with 3-fold excess of the first amino acid (cysteine), HOBt-Cl and DIC for 4 hours in 9:1 v/v CH₂Cl₂/dimethylformamide (DMF) using catalytic amounts of 4-dimethylaminopyridine (DMAP). The resin was then capped with two equivalents of acetic anhydride and pyridine for 30 minutes to block any unreacted hydroxyl groups on the resin. These steps were followed by three washes with methylene chloride (DCM) and DMF, consecutively.

After each coupling reaction, 9-fluorenylmethoxycarbonyl (Fmoc) groups were removed by two 10-minute incubations with 20% (v/v) piperidine in DMF. The resin was then washed twice with DMF prior to adding the next amino acid. Each amino acid was reacted in 3-fold excess 2-(1H-benzotriazol-1-yl)-1,1,3,3-tetramethyluronium hexafluorophosphate (HBTU)/N-methylmorpholine (NMM) for 30 minutes, followed by a double coupling with 3-fold excess benzotriazol-1-yl-oxytripyrrolidinophosphonium hexafluorophosphate (PyBOP)/N-methylmorpholine (NMM) for 30 minutes. All amino acids were added according to the conditions above. Standard Fmoc-protected amino acids with acid-sensitive side chain protecting groups were used, unless otherwise noted. Thereafter, tyrosine or the peptide sequence shown in Table 1 was added onto the peptide using the solid-phase procedures listed above using an automated peptide synthesizer (Focus XC, AAPPTec). Upon synthesis completion, the terminal Fmoc was removed using the aforementioned conditions, after which the resin was washed three times with DMF, three times with DCM, and twice with methanol, and then dried with argon gas.

The dried resin with the peptide was cleaved using 95:2.5:2.5 trifluoroacetic acid/water/triisopropylsilane and excess tris(2-carboxyethyl)phosphine (TCEP) for 2 hours. The peptide was then precipitated from the cleavage solution using 10 times the volume of cold diethyl ether. The solution was spun at 2,000 relative centrifugal force (RCF) for five minutes and then decanted. The pellet was then desiccated and submitted to analytical liquid chromatography-mass spectrometry (1220 LC; 6130 MS, Agilent) for confirmation of synthesis. The crude peptide was dissolved in a mixture of DMF and water and purified via preparative reversed-phase high-performance liquid chromatography (1290, Agilent, Santa Clara, CA). A C-18 column with a 0-50% ammonium acetate: acetonitrile mobile phase for 40 minutes was used to purify the 2,2,6,6-tetramethylpiperidine (TMP). The fraction that contained only pure payloads as assessed by analytical liquid chromatography-mass spectrometry (1220 LC; 6130 MS, Agilent, Santa Clara, CA) was lyophilized (FreeZone, LABCONCO, Kansas City, MO) and stored as lyophilized powder at −20° C. until it was coupled with targeting ligands.

The following substitutions were introduced into residues 1-46 of parathyroid hormone-related protein (PTHrP) (SEQ ID NO: 1): Glu22, Glu25, Leu23, Leu28, Leu31, Lys26, Lys30, and Aib29 in accordance with methods known in the art. These substitutions enhanced peptide stability, induced greater bone density in patients with osteoporosis, and expanded the window of maximal anabolic activity without increasing toxicity. To maximize signaling of the anabolic peptide upon adsorption to exposed hydroxyapatite, the C-terminus of this PTHrP fragment was conjugated to a linear peptide of 20 D-glutamic acid (E) residues (D-Glu20 or DE20) using standard solid phase peptide chemistry, yielding a final fusion protein (SEQ ID NO: 2) in 19% overall yield and final purity of 94% as evidenced by high pressure liquid chromatography (HPLC) and mass spectrometry.

### Example 2: Synthesis of (Linear) Osteotropic Peptides

Targeting ligand peptides were all synthesized to achieve the appropriate length, amino acid composition and enantiomeric stereochemistry, as indicated by their names according to the solid phase synthesis methods described above. While still on the resin, the N-terminal amines were deprotected as described above, and the resin was reacted in DMF with 3-fold maleimide propionic acid, 3-fold excess benzotriazol-1-yloxytripyrrolidinophosphonium hexafluorophosphate (PYBOP), HOBt-Cl and 5-fold excess N,N-diisopropylethylamine (DIPEA) for 4 hours. The peptides were then coupled to the cysteine-containing peptides using maleimide chemistry in phosphate-buffered saline (PBS) containing 10-fold excess TCEP for 24 hours at room temperature. The targeting payload conjugates were then cleaved, deprotected, and purified as described above.

### Example 3: Synthesis of (Branched) Osteotropic Peptides

Briefly, branched targeting ligands were synthesized using solid-phase peptide synthesis under a stream of argon. 2-chlorotrityl resin (0.6 mmol/g) was loaded at 0.6 mmol/g with Nα,Nε-di-Fmoc-L-lysine for 60 minutes in DCM and DIPEA. The resin was then capped with four washes of MeOH, followed by three washes with DCM and DMF, consecutively. The branched chain was then synthesized as described above. The N-terminal Fmoc was retained and the peptide was subjected to a soft cleavage in 1:1:8 mixture of acetic acid/tetrafluoroethylene (TFE)/DCM for 30 minutes. The cleavage solution was evaporated under reduced pressure and the terminal carboxylic acid was conjugated with 3-fold excess N-(2-aminoethyl)maleimide, 3-fold excess PYBOP and HOBt-Cl and 5-fold excess DIPEA in DCM for 4 hours. The acid-sensitive protecting groups were then deprotected by a two-hour incubation in 95:2.5:2.5 trifluoroacetic acid/water/triisopropylsilane. The peptide was then precipitated with 10 volumes of cold diethyl ether, and the terminal Fmoc was deprotected by a 15-minute incubation with 20% (v/v) piperidine in DMF followed by a precipitation in cold diethyl ether. The resulting crude product was purified via preparative reversed-phase high-performance liquid chromatography (1290, Agilent) as described above. Finally, the purified targeting ligand was conjugated with different payloads via maleimide coupling also as described above.

### Example 4: Acidic Oligopeptide Targeting

Rats are the smallest animals that spinal fusion surgeries can be modeled on. The posterolateral lumbar fusion model in rats is the most useful and easily accessible site to perform the surgery. This model is also the easiest in which to measure bone growth over time.

Female Sprague Dawley rats were subjected to bilateral spinal fusion of the transverse process of the L4 and L5 vertebrae by insertion of a mineralized collagen sponge. The rats were injected with 50456, a near infrared (IR) fluorescent dye, conjugated to an acidic oligopeptide (AOP) targeting moiety two weeks post-surgery. Twenty-four hours post-injection, the rats were imaged with a spectral imaging system (AMI) for 1 second with excitation at 745 nM and emission collected at 810 nM. Imaging confirmed localization of the 50456 conjugate to the spine and the kidneys, which are the excretion pathway for the compounds (see, e.g., FIG. 3).

### Example 5: Bilateral Posterolateral Lumbar Surgery

A bilateral posterolateral lumbar spinal fusion was performed under aseptic conditions using isoflurane as an anesthetic. The skin surrounding the L2-L6 region on the back was shaved and disinfected using a Betadine solution followed by an alcohol pad. A 3-cm incision extending from the L3 to L6 vertebrae was made in the skin in this prepared area. The muscle on the vertebral body between the spinous process and the transverses process was bluntly dissected away from the vertebral body to expose the transverses processes.

Using a low-speed burr, the L4 and L5 transverse processes cortical bone was removed in addition to the facia joint between the two vertebrae. The burr removed the surface layer of the bone. This process is known as decortication and was done to allow for direct regeneration of bone tissue. Following decortication, a drug-soaked collagen sponge was placed on each side of the vertebrae. A 5×7.5-mm collagen sponge was soaked to saturation in the drugs for 10 minutes. The collagen sponge (RCM6 membranes from ACE surgical supplies) served as a scaffold to aid in the fusion of the vertebrae. Mineralized collagen scaffolds (supplied by Houston Methodist, Houston, TX) and collagen scaffolds wrapped around anorganic bone granules (InterOss bone granules; SigmaGraft Inc. Biomaterials, Fullerton, CA) were also separately used. The inter-muscle was closed with resorbable sutures. The exterior wound was then sutured closed using a nylon suture. Subcutaneous buprenorphine (0.05-0.1 mg/kg) was then administered directly after the operation and was repeated every 12 hours for the next 3 days post-surgery.

### Example 6: Micro-CT and AMI Scan

Fracture healing was assessed using micro-CT (Scanco Medical Ag or PerkinElmer). The anesthetized animal was scanned for two minutes with the PerkinElmer Quantum FX micro-CT at high resolution with a voxel size of 10 μM at 90 kV and 88 uA and an Al 0.5 mm+Cu 0.06 mm filter. The scanning detector rate was 117 fps. The images were analyzed in ImageJ using the BoneJ package. Morphometric parameters were quantified in an ROI that included just the fracture callus. Trabecular thickness (Tb.Th.), trabecular spacing (Tb. Sp.), total volume (TV), and volume of calcified callus (BV) were calculated. Statistical analysis was performed using a one-way analysis of variance (ANOVA) and a Dunnett's post-hoc analysis with significance reported at the P value of 0.05. All animal experiments were performed in accordance with protocols approved by Purdue University's Institutional Animal Care and Use Committee (IACUC). Rats were scanned in AMI after being anesthetized with isoflurane.

### Example 7: Spinal Fusion Targeting

The ability to localize compounds selectively to the spinal fusion site over the rest of the body from a subcutaneous injection was demonstrated for the three main methods of spinal fusion: collagen sponge, mineralized collagen, and bone grafts. The results are shown in FIGS. 2-4.

### Example 8: Spinal Fusion Targeting

Rats were assigned to one of 3 treatments: saline as a negative control, 10 μg of BMP2 (clinically approved drug; current standard of care) as a positive control, or AbaloDE20 (a compound of the present disclosure). The preliminary results from week 5 are shown in FIGS. 14A-C.

### Example 9: Synthesis of Near Infrared (NIR) Dye Conjugates

A maleimide derivative of the NIR fluorescent dye, 50456, was prepared for use in labeling of the bone fracture targeting ligands described above. It was synthesized as shown in Scheme I (below). For this purpose, 50456, N-Boc-tyramine and potassium hydroxide (KOH) were mixed in a flask containing dimethylsulfoxide (DMSO) to dissolve solids and the solution was stirred at 60° C. under argon for 1.2 hours. The resulting solution was precipitated with cold ethyl acetate and, after vigorous agitation, was centrifuged at 3,000 rpm for 3 minutes. The dark green solid was dried in a vacuum desiccator overnight and deprotected in 40% trifluoroacetic acid (TFA)/DCM for 30 minutes before being concentrated in vacuo to remove all TFA and DCM. The crude solid was then dissolved in water and subjected to preparative reversed-phase high-performance liquid chromatography (1290, Agilent, Santa Clara, CA) purification. Pure fractions were concentrated in vacuo and lyophilized. To derivatize with maleimide, the solid was dissolved in DMSO together with N-succinimidyl 3-maleimidopropionate and DIPEA and stirred under argon atmosphere for one hour before purification via preparative reversed-phase high-performance liquid chromatography (1290, Agilent, Santa Clara, CA) as described above. Deca-aspartic acid (L and D)-targeting ligand with an N-terminal cysteine were prepared and purified as described previously. For conjugation of deca-aspartic acid cysteine to 50456-maleimide, 50456-maleimide was dissolved in DMSO in a flask degassed with argon, followed by the addition of Asp₁₀-Cys to the solution with stirring. The mixture was stirred at room temperature for 2.5 hours before purification with preparative reversed-phase high-performance liquid chromatography (1290, Agilent, Santa Clara, CA). The purified and lyophilized product appeared as a green fluffy solid. Synthesis of (D)Asp₁₀-50456 conjugate followed the same procedure as described for (L)Asp₁₀-50456, except that D-aspartic acid was used for the synthesis of (D)Asp₁₀.

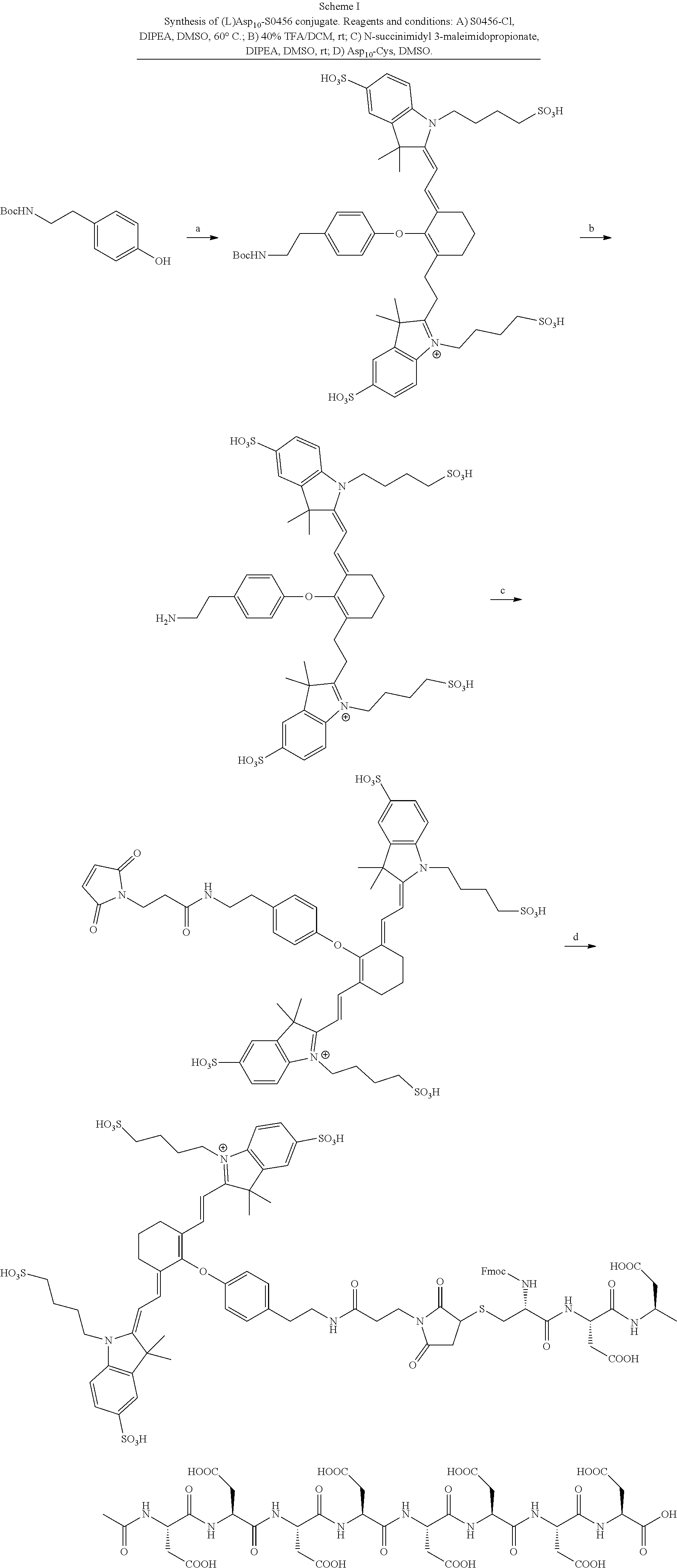

It is recognized that various modifications are possible within the scope of the claimed invention. Thus, it should be understood that, although the present invention has been specifically disclosed in the context of preferred embodiments and optional features, those skilled in the art may resort to modifications and variations of the concepts disclosed herein. Such modifications and variations are considered to be within the scope of the invention as claimed herein.

1. A method of treating a spinal fusion of a patient in need thereof, the method comprising administering to the patient a therapeutically effective amount of a compound having a structure of Formula (I):
X-Y-Z Formula (I),
or a pharmaceutically acceptable salt thereof,
wherein:
X is a bone anabolic agent;
Y is absent or a linker; and
Z is an osteotropic ligand,
thereby treating the spinal fusion of the patient.

2. The method of claim 1, wherein Y is a releasable linker or a non-releasable linker.

3. The method of claim 1, wherein X is abaloparatide, preptin, integrin 5 beta 1 (ITGA), dasatinib, parathyroid hormone (PTH), parathyroid hormone related protein (PTHrP), or a derivative or fragment of any of the foregoing having bone anabolic activity.

4. The method of any one of claims 1-3, wherein X is abaloparatide.

5. The method of claim 1, wherein Z is an acidic oligopeptide (AOP) or another hydroxyapatite binding molecule.

6. The method of claim 1, wherein Z is an AOP comprising at least 4 amino acid residues.

7. The method of any one of claims 1-3, 5, and 6, wherein Z is an AOP comprising 4 to 20 amino acid residues.

8. The method of any one of claims 1-3, 5, and 6, wherein the bone anabolic agent is a parathyroid hormone (PTH), a PTH-related protein (PTHrP), or a derivative or fragment of either of the foregoing having bone anabolic activity.

9. The method of any one of claims 1-3, 5, and 6, wherein the bone anabolic agent is abaloparatide or a derivative or fragment thereof having bone anabolic activity.

10. The method of any one of claims 1-3, 5, and 6, wherein Z is a linear chain of amino acid residues.

11. The method of claim 1, wherein Z is an AOP comprising at least 4 glutamic acid amino acid residues or 4 aspartic acid amino acid residues.

12. The method of any one of claims 1-3, 5, 6, and 11, wherein Z comprises at least 4 amino acid residues having the either D or L chirality.

13. The method of any one of claims 1-3, 5, 6, and 11, wherein Z comprises at least 4 amino acid residues having D chirality.

14. The method of any one of claims 1-3, 5, 6, and 11, wherein Z comprises at least 4 glutamic acid amino acid residues, at least 4 aspartic acid amino acid residues, or at least 4 glutamic acid amino acid residues and at least 4 aspartic acid amino acid residues.

15. The method of any one of claims 1-3, 5, 6, and 11, wherein Z comprises 4 to 20 D-glutamic acid amino acid residues and/or 4 to 20 D-aspartic acid amino acid residues.

16. The method of any one of claims 1-3, 5, 6, and 11, wherein Z comprises a mixture of glutamic acid amino acid residues and aspartic acid amino acid residues.

17. The method of claim 1, wherein Z comprises at least 10 repeating D-glutamic acid amino acid residues (DE10).

18. The method of any one of claims 1-3, 5, 6, 11, and 17, wherein Z comprises at least 15 repeating D-glutamic acid amino acid residues (DE15) or at least 20 repeating D-glutamic acid amino acid residues (DE20).

19. The method of any one of claims 1-3, 5, 6, 11, and 17, wherein Z is DE10 or DE20.

20. The method of any one of claims 1-3, 5, 6, 11, and 17, wherein X is abaloparatide, ITGA, dasatinib, PTH, PTHrP, or a derivative or fragment of any one of the foregoing having bone anabolic activity; and Z is DE20.

21. The method of any one of claims 1-3, 5, 6 and 11, wherein Z comprises 4 to 75 acidic amino acid residues.

22. The method of any one of claims 1-3, 5, 6 and 11, wherein Z comprises 4 to 75 D-glutamic acid amino acid residues.

23. The method of any one of claims 1-3, 5, 6 and 11, wherein Z comprises 8 to 30 acidic amino acid residues.

24. The method of any one of claims 1-3, 5, 6 and 11, wherein Z comprises 8 to 30 D-glutamic acid amino acid residues.

25. The method of any one of claims 1-3, 5, 6, 11, and 17, wherein Y is a non-releasable linker containing at least one carbon-carbon bond, at least one amide bond, or at least one carbon-carbon bond and at least one amide bond.

26. The method of any one of claims 1-3, 5, 6, 11, and 17, wherein Y is a releasable linker.

27. The method of any one of claims 1-3, 5, 6, 11, and 17, wherein Y is a releasable linker containing at least one disulfide bond, at least one ester, at least one protease-specific amide bond, or a combination of the foregoing.

28. The method of claim 1, wherein:
X is abaloparatide, ITGA, dasatinib, PTH, PTHrP, or a derivative or fragment of any of the foregoing having bone anabolic activity;
Y is a non-releasable oligopeptide linker; and
Z is DE20.

29. The method of claim 1, wherein X is abaloparatide or a derivative or fragment thereof, Y is a releasable oligopeptide linker comprising at least one protease-specific amide bond, and Z is DE20.

30. The method of claim 1, wherein the compound has at least 75% sequence identity or more, at least 85% sequence identity or more, at least 90% sequence identity or more, or at least 95% sequence identity or more to SEQ ID NO: 1.

31. The method of claim 1, wherein the compound has at least 75% sequence identity or more, at least 85% sequence identity or more, at least 90% sequence identity or more, or at least 95% sequence identity or more to SEQ ID NO: 2.

32. A kit for treating a spinal fusion in a patient in need thereof and/or targeting a therapeutic agent or a diagnostic agent to a spinal fusion in the patient, the kit comprising:
(a) a compound having a structure of Formula (I):
X-Y-Z Formula (I),
or a pharmaceutically acceptable salt thereof,
wherein:
X is a therapeutic agent for treating a spinal fusion in the patient or a diagnostic agent,
Y is absent or a linker, and
Z is an osteotropic ligand; and
(b) a collagen sponge, a mineralized collagen, or a bone graft.

33. The kit of claim 32, wherein X comprises a bone anabolic agent and wherein administration of a therapeutically effective amount of the compound having a structure of Formula (I) or a pharmaceutically acceptable salt thereof to the patient treats the spinal fusion.

34. The kit of claim 33, wherein X is a therapeutic agent selected from the group consisting of abaloparatide, integrin 5 beta 1 (ITGA), dasatinib, parathyroid hormone (PTH), parathyroid hormone related protein (PTHrP), and a derivative or fragment of any of the foregoing having bone anabolic activity.

35. The kit of claim 32, wherein X comprises a diagnostic agent and administration of a therapeutically effective amount of a compound having a structure of Formula (I) or a pharmaceutically acceptable salt thereof to the patient identifies a spinal fusion if present.

36. The kit of any one of claims 32-35, wherein Y is a releasable linker or a non-releasable linker.

37. The kit of any one of claims 32-35, wherein Y is a non-releasable linker containing at least one carbon to carbon bond, at least one amide bond, or at least one carbon to carbon bond and at least one amide bond.

38. The kit of any one of claims 32-35, wherein Z is an acidic oligopeptide (AOP).

39. The kit of any one of claims claim 32-35, wherein Z is an AOP comprising at least 4 amino acid residues.

40. The kit of any one of claims 32-35, wherein the AOP comprises 4 to 20 amino acid residues.

41. The kit of any one of claims 32-35, wherein X is a bone anabolic agent that is a parathyroid hormone (PTH), a PTH receptor protein (PTHrP), or a derivative or fragment of either of the foregoing having bone anabolic activity.

42. The kit of claim 32, wherein X is a therapeutic agent that is abaloparatide or a derivative or fragment thereof having bone anabolic activity.

43. A pharmaceutical composition comprising a compound having a structure of Formula (I):
X-Y-Z Formula (I),
or a pharmaceutically acceptable salt thereof,
wherein:
X is a therapeutic agent or a diagnostic agent,
Y is absent or a linker, and
Z is an osteotropic ligand.

44. The pharmaceutical composition of claim 43 further comprising at least one pharmaceutically acceptable carrier or excipient.

45. The pharmaceutical composition of claim 43 or 44 formulated for subcutaneous administration to the patient.

46. The pharmaceutical composition of claim 43, wherein X is a therapeutic agent that is a bone anabolic agent.

47. The pharmaceutical composition of claim 43, wherein X is a diagnostic agent that is an imaging agent.

48. The pharmaceutical composition of claim 43, wherein Y is a releasable linker or a non-releasable linker.

49. The pharmaceutical composition of any one of claims 43, 44 and 46, wherein X is abaloparatide, preptin, integrin 5 beta 1 (ITGA), dasatinib, parathyroid hormone (PTH), parathyroid hormone related protein (PTHrP), or a derivative or fragment of any of the foregoing having bone anabolic activity.

50. The pharmaceutical composition of claim any one of claims 43, 44 and 46, wherein Z is an acidic oligopeptide (AOP) or another hydroxyapatite binding molecule.

51. The pharmaceutical composition of claim any one of claims 43, 44 and 46, wherein Z is an AOP comprising at least 4 amino acid residues.

52. The pharmaceutical composition of any one of claims 43, 44 and 46, wherein Z is an AOP comprising 4 to 20 amino acid residues.

53. The pharmaceutical composition of any one of claims 43, 44 and 46, wherein the bone anabolic agent is abaloparatide or a derivative or fragment thereof having bone anabolic activity.

54. The pharmaceutical composition of any one of claims 43, 44 and 46, wherein Z is a linear chain of amino acid residues.

55. The pharmaceutical composition of any one of claims 43, 44 and 46, wherein Z comprises 4 to 20 D-glutamic acid amino acid residues and/or 4 to 20 D-aspartic acid amino acid residues.

56. The pharmaceutical composition of claim 43, wherein Z comprises at least 10 repeating D-glutamic acid amino acid residues (DE10).

57. The pharmaceutical composition of any one of claims 43, 44 and 46, wherein Z comprises at least 15 repeating D-glutamic acid amino acid residues (DE15) or at least 20 repeating D-glutamic acid amino acid residues (DE20).

58. The pharmaceutical composition of any one of claims 43, 44, 46, and 56, wherein:
X is abaloparatide, ITGA, dasatinib, PTH, PTHrP, or a derivative or fragment of any one of the foregoing having bone anabolic activity;
Y is a non-releasable linker; and
Z is DE20.

59. The pharmaceutical composition of any one of claims 43, 44, 46, and 56, wherein Y is a non-releasable linker containing at least one carbon-carbon bond, at least one amide bond, or at least one carbon-carbon bond and at least one amide bond.

60. The pharmaceutical composition of any one of claims 43, 44, 46, and 56, wherein Y is a releasable linker.

61. The pharmaceutical composition of any one of claims 43, 44, 46, and 56, wherein Y is a releasable linker containing at least one disulfide bond, at least one ester, at least one protease-specific amide bond, or a combination of the foregoing.

62. The pharmaceutical composition of claim 43, wherein the compound has at least 75% sequence identity or more, at least 85% sequence identity or more, at least 90% sequence identity or more, or at least 95% sequence identity or more to SEQ ID NO: 1.

63. The pharmaceutical composition of claim 43, wherein the compound has at least 75% sequence identity or more, at least 85% sequence identity or more, at least 90% sequence identity or more, or at least 95% sequence identity or more to SEQ ID NO: 2.

64. A method for localizing a therapeutic agent or a diagnostic agent to a spinal fusion site in a patient, the method comprising administering to the patient a therapeutically effective amount of a compound having a structure of Formula (I):
X-Y-Z Formula (I),
or a pharmaceutically acceptable salt thereof,
wherein:
X is a therapeutic agent for treating a spinal fusion in the patient or a diagnostic agent;
Y is absent or a linker; and
Z is an osteotropic ligand.

65. The method of claim 64, wherein Y is a releasable linker or a non-releasable linker.

66. The method of claim 64, wherein Y is a non-releasable linker.

67. The method of claim 64 or 65, wherein X is a diagnostic agent that is a fluorescent dye.

68. The method of claim 64, wherein X is a therapeutic agent for treating a spinal fusion that is a bone anabolic agent.

69. The method of claim 64, wherein Z is an acidic oligopeptide (AOP) or another hydroxyapatite binding molecule.

70. The method of claim 64, wherein Z is an AOP comprising at least 4 amino acid residues.

71. The method of any one of claims 64-66 and 68-70, wherein X is a therapeutic agent that is abaloparatide, integrin 5 beta 1 (ITGA), dasatinib, parathyroid hormone (PTH), parathyroid hormone related protein (PTHrP), or a derivative or fragment of any of the foregoing having bone anabolic activity.

72. The method of any one of claims 64-66 and 68-70, wherein X is a therapeutic agent that is a PTH, a PTHrP, or a derivative or fragment of either of the foregoing having bone anabolic activity.

73. The method of any one of claims 64-66 and 68-70, wherein X is a therapeutic agent that is abaloparatide or a derivative or fragment thereof having bone anabolic activity.

74. The method of any one of claims 64-66 and 68-70, wherein Z is a linear chain of amino acid residues.

75. The method of any one of claims 64-66 and 68-70, wherein Z comprises 4 to 20 D-glutamic acid amino acid residues, 4 to 20 D-aspartic acid amino acid residues, or 4 to 20 D-glutamic acid amino acid residues and 4 to 20 D-aspartic acid amino acid residues.

76. The method of claim 64, wherein Z comprises at least 10 repeating D-glutamic acid amino acid residues (DE10).

77. The method of any one of claims 64-66, 68-70, and 76, wherein Z comprises at least 15 repeating D-glutamic acid amino acid residues (DE15) or at least 20 repeating D-glutamic acid amino acid residues (DE20).

78. The method of any one of claims 64-66, 68-70, and 76, wherein X is a therapeutic agent that is abaloparatide or a derivative or fragment thereof having bone anabolic activity and Z is DE20.

79. The method of claim 64, wherein the compound has at least 75% sequence identity or more, at least 85% sequence identity or more, at least 90% sequence identity or more, or at least 95% sequence identity or more to SEQ ID NO: 1.

80. The method of claim 64, wherein the compound has at least 75% sequence identity or more, at least 85% sequence identity or more, at least 90% sequence identity or more, or at least 95% sequence identity or more to SEQ ID NO: 2.
